(19) United States
(12) Patent Application Publication
(10) Pub. No.: US 2014/0005096 A1
(43) Pub. Date: Jan. 2, 2014

(54) METHODS OF TREATING INFLAMMATION

(71) Applicant: Carolus Therapeutics, Inc., La Jolla, CA (US)

(72) Inventors: Joshua Robert SCHULTZ, Ballston Lake, NY (US); Benedikt VOLLRATH, San Diego, CA (US); Court TURNER, San Diego, CA (US)

(73) Assignee: Carolus Therapeutics, Inc., La Jolla, CA (US)

(21) Appl. No.: 13/942,487

(22) Filed: Jul. 15, 2013

Related U.S. Application Data
(63) Continuation of application No. 12/574,541, filed on Oct. 6, 2009, now abandoned.
(60) Provisional application No. 61/121,779, filed on Dec. 11, 2008.
(60) Provisional application No. 61/118,938, filed on Dec. 1, 2008.
(60) Provisional application No. 61/115,450, filed on Nov. 17, 2008.
(60) Provisional application No. 61/113,979, filed on Nov. 12, 2008.
(60) Provisional application No. 61/103,182, filed on Oct. 6, 2008.

(51) Int. Cl. C07K 14/52; A61K 45/06; A61K 38/19

(52) U.S. Cl. CPC C07K 14/523; A61K 38/19; A61K 45/06
USPC 514/1.4; 530/300; 530/326; 514/13.8; 514/1.9; 514/1.5; 514/6.9; 514/7.3; 514/4.8; 514/1.7; 514/1.8; 514/7.4

(57) ABSTRACT

Disclosed herein, in certain embodiments, are peptides for use in inhibiting the interactions of PF4 and RANTES. Further disclosed herein, are methods for treating an inflammatory disease, disorder, condition, or symptom. In some embodiments, the method comprises co-administering an agent that inhibits the interactions of PF4 and RANTES and a second active agent.

### CROSS-REFERENCE

This application is a continuation of U.S. patent application Ser. No. 12/574,541 filed on Oct. 6, 2009, which claims the benefit of U.S. Provisional Application No. 61/103,182, filed Oct. 6, 2008; U.S. Provisional Application No. 61/113,979, filed Nov. 12, 2008; U.S. Provisional Application No. 61/115,450, filed Nov. 17, 2008; U.S. Provisional Application No. 61/118,938, filed Dec. 1, 2008; and U.S. Provisional Application No. 61/121,779, filed Dec. 11, 2008, all of which are incorporated herein by reference.

### BACKGROUND OF THE INVENTION

Inflammatory diseases, disorders, conditions and symptoms are characterized, in part, by the migration of lymphocytes and monocytes into the affected tissue. The migration of lymphocytes and monocytes induces tissue damage and exacerbates inflammatory diseases, disorders, conditions and symptoms.

RANTES (also known as CCL5) and PF4 are pro-inflammatory chemokines. In certain instances, they are secreted by an activated platelet in response to an inflammation or tissue injury. In certain instances, RANTES and PF4 induce chemotaxis in nearby leukocytes (e.g. monocytes) along their gradients.

### SUMMARY OF THE INVENTION

There is a need for new methods of treating inflammatory diseases, disorders, conditions (e.g., atherosclerosis) and symptoms that do not interfere with (a) non-inflammatory processes or (b) desired-inflammatory processes. The inventors have discovered that undesired and harmful inflammation can be treated by inhibiting the interactions of PF4 and RANTES. Further, the inventors have discovered that targeting precise regions of PF4 and RANTES will inhibit the ability of the ligands to bind to each other and their receptors (thus, preventing undesired inflammation) without affecting other (e.g., desired and beneficial) interactions of PF4 and RANTES.

There is also a need to develop methods and compositions for treating inflammatory diseases, disorders, conditions that combine (a) a first agent that inhibits inflammation with (b) a second agent that otherwise treats an inflammatory disease, disorder, condition but results (or has been shown to result) in undesired inflammation (e.g., myositis).

Disclosed herein, in certain embodiments, is an isolated peptide, its pharmacologically acceptable salts, derivatives, and conjugates, characterized in that the peptide has an amino acid sequence SEQ ID NO: 1, as indicated below:

(SEQ ID NO: 1)
C-X1-X2-YFYTS-X3-X4-X5-X6-X7-X8-X9-X10-X11-X12-
X13-X14-X15-C

where:
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine;
X15 is chosen from the group containing arginine, lysine, alanine, glutamine, histidine and asparagine, or an amino acid deletion.
In some embodiments, the peptide has an amino acid sequence SEQ ID NO: 2, as indicated below:

C-KEYFYTSGKCSNPAVVFVTR-C.

In some embodiments, the peptide has an amino acid sequence SEQ ID NO: 3, as indicated below:

C- KEYFYTSSKCSNLAVVFVTR-C.

In some embodiments, the peptide has an amino acid sequence SEQ ID NO: 4, as indicated below:

C- QEYFYTSSKCSMAAVVFITR-C.

In some embodiments, the peptide has an amino acid sequence SEQ ID NO: 13, as indicated below:

(SEQ ID NO 13)
C-KEYFYTSSKSSNLAVVFVTR-C.

In some embodiments, the peptide has an amino acid sequence SEQ ID NO: 14, as indicated below:

(SEQ ID NO 14)
CSFKGTTVYALSNVRSYSFVKC.

In some embodiments, the peptide has an amino acid sequence SEQ ID NO: 15, as indicated below:

(SEQ ID NO 15)
CSFKGTNVYALTKVRSYSFVSC.

In some embodiments, the peptide is selected from: SSKSSNLAVVFVTRCCKEYFYT (SEQ ID NO 45); SKSSNLAVVFVTRCCKEYFYTS (SEQ ID NO 46); KSSNLAVVFVTRCCKEYFYTSS (SEQ ID NO 47); SSNLAVVFVTRCCKEYFYTSSK (SEQ ID NO 48); SNLAVVFVTRCCKEYFYTSSKS (SEQ ID NO 49); NLAVVFVTRCCKEYFYTSSKSS (SEQ ID NO 50); SFKGTTVYALSNVRSYSFVKCC (SEQ ID NO 51); FKGTTVYALSNVRSYSFVKCCS (SEQ ID NO 52); SNVRSYSFVKCCSFKGTTVYAL (SEQ ID NO 53); NVRSYSFVKCCSFKGTTVYALS (SEQ ID NO 54); SYSFVKCCSFKGTTVYALSNVR (SEQ ID NO 55); YSFVKCCSFKGTTVYALSNVRS (SEQ ID NO 56); SFVKCCSFKGTTVYALSNVRSY (SEQ ID NO 57); FVKCCSFKGTTVYALSNVRSYS (SEQ ID NO 58); or a combination thereof.

Disclosed herein, in certain embodiments, is a method of treating an inflammatory disease, disorder, condition, or symptom, comprising administering to an individual in need thereof a therapeutically-effective amount of agent that inhibits interactions between RANTES and Platelet Factor 4.

In some embodiments, the active agent specifically binds to the RANTES interacting domain of PF4. In some embodiments, the active agent is an isolated peptide that has the amino acid sequence SEQ ID NO: 1, as indicated below:

C-X1-X2-YFYTS-X3-X4-X5-X6-X7-X8-X9-X10-X11-X12-
X13-X14-X15-C

where:
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine;
X15 is chosen from the group containing arginine, lysine, alanine, glutamine, histidine and asparagine, or an amino acid deletion.
In some embodiments, the active agent is an isolated peptide that has the amino acid sequence SEQ ID NO: 2, as indicated below:

C-KEYFYTSGKCSNPAVVFVTR-C.

In some embodiments, the active agent is an isolated peptide that has the amino acid sequence SEQ ID NO: 3, as indicated below:

C- KEYFYTSSKCSNLAVVFVTR-C.

In some embodiments, the active agent is an isolated peptide that has the amino acid sequence SEQ ID NO: 4, as indicated below:

C- QEYFYTSSKCSMAAVVFITR-C.

In some embodiments, the active agent is an isolated peptide that has the amino acid sequence SEQ ID NO: 13, as indicated below:

(SEQ ID NO 13)
C-KEYFYTSSKSSNLAVVFVTR-C.

In some embodiments, the active agent is an isolated peptide that has the amino acid sequence SEQ ID NO: 14, as indicated below:

(SEQ ID NO 14)
CSFKGTTVYALSNVRSYSFVKC.

In some embodiments, the active agent is an isolated peptide that has the amino acid sequence SEQ ID NO: 15, as indicated below:

(SEQ ID NO 15)
CSFKGTNVYALTKVRSYSFVSC.

In some embodiments, the active agent is selected from: SSKSSNLAVVFVTRCCKEYFYT (SEQ ID NO 45); SKSSNLAVVFVTRCCKEYFYTS (SEQ ID NO 46); KSSNLAVVFVTRCCKEYFYTSS (SEQ ID NO 47); SSNLAVVFVTRCCKEYFYTSSK (SEQ ID NO 48); SNLAVVFVTRCCKEYFYTSSKS (SEQ ID NO 49); NLAVVFVTRCCKEYFYTSSKSS (SEQ ID NO 50); SFKGTTVYALSNVRSYSFVKCC (SEQ ID NO 51); FKGTTVYALSNVRSYSFVKCCS (SEQ ID NO 52); SNVRSYSFVKCCSFKGTTVYAL (SEQ ID NO 53); NVRSYSFVKCCSFKGTTVYALS (SEQ ID NO 54); SYSFVKCCSFKGTTVYALSNVR (SEQ ID NO 55); YSFVKCCSFKGTTVYALSNVRS (SEQ ID NO 56); SFVKCCSFKGTTVYALSNVRSY (SEQ ID NO 57); FVKCCSFKGTTVYALSNVRSYS (SEQ ID NO 58); or a combination thereof. In some embodiments, the inflammatory disease, disorder or condition is Atherosclerosis; Abdominal aortic aneurysm (AAA) disease; Acute disseminated encephalomyelitis; Moyamoya disease; Takayasu disease; Acute coronary syndrome; Cardiac-allograft vasculopathy; Pulmonary inflammation; Acute respiratory distress syndrome; Pulmonary fibrosis; Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome; Multiple sclerosis; Myasthenia gravis; Myocarditis; Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder; Endotoxin shock; Septic shock; Rheumatoid spondylitis; Ankylosing spondylitis; Gouty arthritis; Polymyalgia rheumatica; Alzheimer's disorder; Parkinson's disorder; Epilepsy; AIDS dementia; Asthma; Adult respiratory distress syndrome; Bronchitis; Cystic fibrosis; Acute leukocyte-mediated lung injury; Distal proctitis; Wegener's granulomatosis; Fibromyalgia; Bronchitis; Uveitis; Conjunctivitis; Psoriasis; Eczema; Dermatitis; Smooth muscle proliferation disorders; Meningitis; Shingles; Encephalitis; Nephritis; Tuberculosis; Retinitis; Atopic dermatitis; Pancreatitis; Periodontal gingivitis; Coagulative Necrosis; Liquefactive Necrosis; Fibrinoid Necrosis; Neointimal hyperplasia; Myocardial infarction; Stroke; organ transplant rejection; influenza, or combinations thereof.

Disclosed herein, in certain embodiments, is a method of treating a disorder of a cardiovascular system, comprising co-administering to an individual in need thereof a synergistic combination of (a) a therapeutically-effective amount of an agent that inhibits the interaction between RANTES and Platelet Factor 4; and (b) a second active agent selected from an agent that treats a cardiovascular disorder. In some embodiments, administration of the second active agent partially or fully results in undesired inflammation. In some embodiments, the second active agent is niacin; a fibrate; a statin; an apolipoprotein A-1 modulator; an ACAT modulator; a CETP modulator; a glycoprotein IIb/IIIa modulator; a P2Y12 modulator; an Lp-PLA2 modulator; an anti-hypertensive; a leukotriene inhibitor; an 5-LO inhibitor; a FLAP inhibitor; or combinations thereof. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; acute coronary syndrome; unstable angina; non-ST segment elevation myocardial infarction; ST segment elevation myocardial infarction; stable angina; Prinzmetal's angina; arteriosclerosis; atherosclerosis; arteriolosclerosis; stenosis; restenosis; venous thrombosis; arterial thrombosis; stroke; transient ischemic attack; peripheral vascular disease; coronary artery disease; hypertension; or combinations thereof.

### DETAILED DESCRIPTION OF THE INVENTION

Disclosed herein, in certain embodiments, are methods and pharmaceutical compositions for modulating a disorder of a cardiovascular system synergistic combination of (a) a therapeutically-effective amount of a first active agent that inhibits inflammation and treats a cardiovascular disorder selected from (1) a modulator of MIF; (2) a modulator of an interaction between RANTES and Platelet Factor 4; or (3) combinations thereof; and (b) a second active agent selected from an agent that treats a cardiovascular disorder (the “cardiovascular disorder agent”).

In some embodiments, the combination is synergistic and results in a more efficacious therapy. In some embodiments, the therapy synergistically treats cardiovascular disorders by (a) targeting multiple pathways that result in (either partially or fully) development of a cardiovascular disorder (e.g., LDL concentrations and the chemotaxis of macrophages) and (b) treating and/or ameliorating undesired inflammation (e.g, myositis) resulting from the cardiovascular disorder agent. In some embodiments, the therapy synergistically treats cardiovascular disorders by targeting multiple pathways that result in (either partially or fully) development of a cardiovascular disorder (e.g., LDL concentrations and the chemotaxis of macrophages).

In some embodiments, the combination rescues a mammal from inflammation partially or fully caused by the cardiovascular disorder agent. In some embodiments, the combination allows (partially or fully) a medical professional to increase the prescribed dosage of the cardiovascular disorder agent. In some embodiments, the combination enables (partially or fully) a medical professional to prescribe the cardiovascular disorder agent (i.e., co-administration rescues the cardiovascular disorder agent).

In some embodiments, the first active agent (i.e., a MIF antagonist and/or a modulator of an interaction between RANTES and Platelet Factor 4), and a statin synergistically treat a CVD by (1) decreasing the chemotaxis of leukocytes, and (2) decreasing (either partially or fully) cholesterol synthesis. In some embodiments, first active agent further treats undesired inflammation resulting from administration of the statin.

In some embodiments, the first active agent (i.e., a MIF antagonist and/or a modulator of an interaction between RANTES and Platelet Factor 4) and a fibrate synergistically treat a CVD by (1) decreasing the chemotaxis of leukocytes, and (2) increasing the concentration of HDL. In some embodiments, the first active agent also decreases any undesired inflammation resulting from administration of the fibrate.

In some embodiments, the first active agent (i.e., a MIF antagonist and/or a modulator of an interaction between RANTES and Platelet Factor 4) and a ApoA1 modulator synergistically treat a CVD by (1) decreasing the chemotaxis of leukocytes, and (2) increasing the concentration of HDL. In some embodiments, the first active agent also decreases any undesired inflammation resulting from administration of the ApoA1 modulator.

In some embodiments, the first active agent (i.e., a MIF antagonist and/or a modulator of an interaction between RANTES and Platelet Factor 4) and a ACAT modulator synergistically treat a CVD by (1) decreasing the chemotaxis of leukocytes, and (2) decreasing (a) the production and release of apoB-containing lipoproteins and (b) foam cell formation. In some embodiments, the first active agent also decreases any undesired inflammation resulting from administration of the ACAT inhibitor.

In some embodiments, the first active agent (i.e., a MIF antagonist and/or a modulator of an interaction between RANTES and Platelet Factor 4) and a CETP modulator synergistically treat a CVD by (1) decreasing the chemotaxis of leukocytes, and (2) decreasing the transfer cholesterol from HDL cholesterol to LDL. In some embodiments, the first active agent also decreases any undesired inflammation resulting from administration of the CETP inhibitor.

In some embodiments, the first active agent (i.e., a MIF antagonist and/or a modulator of an interaction between RANTES and Platelet Factor 4) and a GP IIb/IIIa receptor antagonist synergistically treat a CVD by (1) decreasing the chemotaxis of leukocytes, and (2) inhibiting platelet aggregation. In some embodiments, the first active agent also decreases any undesired inflammation resulting from administration of the GP IIb/IIIa receptor antagonist.

In some embodiments, the first active agent (i.e., a MIF antagonist and/or a modulator of an interaction between RANTES and Platelet Factor 4) and a P2Y12 receptor antagonist synergistically treat a CVD by (1) decreasing the chemotaxis of leukocytes, and (2) inhibiting platelet aggregation. In some embodiments, the first active agent also decreases any undesired inflammation resulting from administration of the P2Y12 receptor antagonist.

In some embodiments, the first active agent (i.e., a MIF antagonist and/or a modulator of an interaction between RANTES and Platelet Factor 4) and a Lp-PLA2 antagonist synergistically treat a CVD by (1) decreasing the chemotaxis of leukocytes, and (2) inhibiting the formation of biologically active products from oxidized LDL. In some embodiments, the first active agent also decreases any undesired inflammation resulting from administration of the Lp-PLA2 antagonist.

### CERTAIN DEFINITIONS

The terms “individual,” “individual,” or “subject” are used interchangeably. As used herein, they mean any mammal (i.e. species of any orders, families, and genus within the taxonomic classification animalia: chordata: vertebrata: mammalia). In some embodiments, the mammal is a human. In some embodiments, the mammal is a non-human. In some embodiments, the mammal is a member of the taxonomic orders: primates (e.g. lemurs, lorids, galagos, tarsiers, monkeys, apes, and humans); rodentia (e.g. mice, rats, squirrels, chipmunks, and gophers); lagomorpha (e.g. hares, rabbits, and pika); erinaceomorpha (e.g. hedgehogs and gymnures); soricomorpha (e.g. shrews, moles, and solenodons); chiroptera (e.g., bats); cetacea (e.g. whales, dolphins, and porpoises); carnivora (e.g. cats, lions, and other feliformia; dogs, bears, weasels, and seals); perissodactyla (e.g. horse, zebra, tapir, and rhinoceros); artiodactyla (e.g. pigs, camels, cattle, and deer); proboscidea (e.g. elephants); sirenia (e.g. manatees, dugong, and sea cows); cingulata (e.g. armadillos); pilosa (e.g. anteaters and sloths); didelphimorphia (e.g. american opossums); paucituberculata (e.g. shrew opossums); microbiotheria (e.g. Monito del Monte); notoryctemorphia (e.g. marsupial moles); dasyuromorphia (e.g. marsupial carnivores); peramelemorphia (e.g. bandicoots and bilbies); or diprotodontia (e.g. wombats, koalas, possums, gliders, kangaroos, wallaroos, and wallabies). In some embodiments, the animal is a reptile (i.e. species of any orders, families, and genus within the taxonomic classification animalia: chordata: vertebrata: reptilia). In some embodiments, the animal is a bird (i.e. animalia: chordata: vertebrata: ayes). None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored prodrugs are those that increase the bioavailability of the agents of this invention when such agents are administered to an individual (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The term “recruiting of monocytes” as described herein includes the migration of monocytes into or out of the endothelium, their attachment and propagation, for example, into endothelial fissures. The attachment of monocytes is also known as monocyte adhesion, or as monocyte arrest when the attachment occurs in shear flow as under physiological conditions, for example, in blood capillaries, microvascular or arterial streamlines.

By the term “polypeptide” is meant synthetic or nonsynthetic peptide compounds, as well as purified, modified fragments of natural proteins, native forms or recombinant peptides or proteins. The term “polypeptide” likewise includes pharmacologically acceptable salts, pharmacologically acceptable derivatives and/or conjugates of the corresponding polypeptide.

Pharmacologically acceptable derivatives include, for example, esters, amides, N-acyl and/or O-acyl derivatives, carboxylated, acetylated, phosphorylated and/or glycosylated polypeptides. Conjugates include, for example, sugar or polyethylene glycol conjugates, biotinylated, radioactively or fluorescently labeled polypeptides.

The term “peptide mimetic”, “mimetic peptide” and “analog” are used herein interchangeably for the purposes of the specifications and claims, to mean a peptide that mimics part or all of the bioactivity of an endogenous protein ligand. In one embodiment, peptide mimetics are modeled after a specific peptide and display an altered peptide backbone, altered amino acids and/or an altered primary amino acid sequence when compared to the peptide of which is was designed to mimic.

As used herein, the terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and immunoglobulin are used interchangeably in the broadest sense. In some embodiments an antibody is part of a larger fusion molecule, formed by covalent or non-covalent association of the antibody with one or more other proteins or peptides.

As used herein, the term “derivative” in the context of a polypeptide or protein, e.g. an antibody, refers to a polypeptide or protein that comprises an amino acid sequence which has been altered by the introduction of amino acid residue substitutions, deletions or additions. The term “derivative” as used herein also refers to a polypeptide or protein which has been modified, i.e., by the covalent attachment of any type of molecule to the antibody. For example, in some embodiments a polypeptide or protein is modified, e.g., by glycosylation, acetylation, pegylation, phosphorylation, amidation, derivatization by protecting/blocking groups, proteolytic cleavage, linkage to a cellular ligand or other protein, etc. In some embodiments, derivatives, polypeptides or proteins are produced by chemical modifications using techniques, including, but not limited to specific chemical cleavage, acetylation, formylation, metabolic synthesis of tunicamycin, etc. In some embodiments a derivative a polypeptide or protein possesses a similar or identical function as the polypeptide or protein from which it was derived.

The terms “full length antibody”, “intact antibody” and “whole antibody” are used herein interchangeably, to refer to an antibody in its substantially intact form, and not antibody fragments as defined below. These terms particularly refer to an antibody with heavy chains contains Fc regions. In some embodiments an antibody variant of the invention is a full length antibody. In some embodiments the full length antibody is human, humanized, chimeric, and/or affinity matured.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures, such as for example, Marks et al., (1992) Biotechnology 10:779-783 that describes affinity maturation by variable heavy chain (VH) and variable light chain (VL) domain shuffling. Random mutagenesis of CDR and/or framework residues is described in: Barbas, et al. (1994) Proc. Nat. Acad. Sci, USA 91:3809-3813; Shier et al., (1995) Gene 169:147-155; Yelton et al., 1995, J. Immunol. 155:1994-2004; Jackson et al., 1995, J. Immunol. 154(7):3310-9; and Hawkins et al, (19920, J. Mol. Biol. 226:889-896, for example.

The terms “binding fragment”, “antibody fragment” or “antigen binding fragment” are used herein, for purposes of the specification and claims, to mean a portion or fragment of an intact antibody molecule, preferably wherein the fragment retains antigen-binding function. Examples of antibody fragments include Fab, Fab′, F(ab′)₂, Fd, Fd′ and Fv fragments, diabodies, linear antibodies (Zapata et al. (1995) Protein Eng. 10: 1057), single-chain antibody molecules, single-chain binding polypeptides, scFv, bivalent scFv, tetravalent scFv, and bispecific or multispecific antibodies formed from antibody fragments.

“Fab” fragments are typically produced by papain digestion of antibodies resulting in the production of two identical antigen-binding fragments, each with a single antigen-binding site and a residual “Fc” fragment. Pepsin treatment yields a F(ab′)2 fragment that has two antigen-combining sites capable of cross-linking antigen. An “Fv” is the minimum antibody fragment that contains a complete antigen recognition and binding site. In a two-chain Fv species, this region consists of a dimer of one heavy- and one light-chain variable domain in tight, non-covalent association. In a single-chain Fv (scFv) species, one heavy- and one light-chain variable domain are covalently linked by a flexible peptide linker such that the light and heavy chains associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although usually at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Methods for producing the various fragments from monoclonal Abs include, e.g., Plückthun, 1992, Immunol. Rev. 130:152-188.

The term “monoclonal antibody” as used herein refers to an antibody obtained from a population of substantially homogeneous antibodies, i.e., the individual antibodies comprising the population are identical except for possible naturally occurring mutations that are present in minor amounts. In some embodiments monoclonal antibodies are made, for example, by the hybridoma method first described by Köhler and Milstein (1975) Nature 256:495, or are made by recombinant methods, e.g., as described in U.S. Pat. No. 4,816,567. In some embodiments monoclonal antibodies are isolated from phage antibody libraries using the techniques described in Clackson et al., Nature 352:624-628 (1991), as well as in Marks et al., J. Mol. Biol. 222:581-597 (1991).

As used herein, the term “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method, for example by immunoassays. Antigenic epitopes need not necessarily be immunogenic.

The phrase “specifically binds” when referring to the interaction between an antibody or other binding molecule and a protein or polypeptide or epitope, typically refers to an antibody or other binding molecule that recognizes and detectably binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react with non-target antigens and/or epitopes. Further, in some embodiments, an antibody that specifically binds, binds through the variable domain or the constant domain of the antibody. For the antibody that specifically binds through its variable domain, it is not aggregated, i.e., is monomeric. A variety of immunoassay formats are used to select antibodies or other binding molecule that are immunoreactive with a particular polypeptide and have a desired specificity. For example, solid-phase ELISA immunoassays, BIAcore, flow cytometry and radioimmunoassays are used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity. “Selective binding”, “selectivity”, and the like refer the preference of a antibody to interact with one molecule as compared to another. Preferably, interactions between antibodies, particularly modulators, and proteins are both specific and selective. Note that in some embodiments a small antibody is designed to “specifically bind” and “selectively bind” two distinct, yet similar targets without binding to other undesirable targets.

### RANTES and Platelet Factor 4 (PF4)

In some embodiments, the methods and compositions disclosed herein inhibit (partially or fully) the activity of RANTES. RANTES (also known as CCL5) is a pro-inflammatory chemokine. In certain instances, it is secreted by an activated platelet in response to an inflammation or tissue injury. In certain instances, RANTES is a ligand for a CCR5 receptor found on the plasma membrane of a target leukocyte (e.g. monocyte). In certain instances, RANTES induces chemotaxis in nearby leukocytes (e.g. monocytes) along a RANTES gradient. In certain instances, RANTES induces the chemotaxis of a leukocyte to the site of an inflammation or tissue injury. In certain instances, the chemotaxis of monocytes along a RANTES gradient results in monocyte arrest (i.e., the deposition of monocytes on epithelium) at the site of injury or inflammation.

In some embodiments, the methods and compositions disclosed herein inhibit (partially or fully) the activity of Platelet Factor 4 (PF4). PF4 (also known as CXCL4) is a chemokine. In certain instances, it is secreted by the alpha granules of an activated platelet during platelet aggregation in response to tissue injury and/or inflammation. In certain instances, PF4 is a ligand for a CXC3 receptor (i.e., CXC3RB). In certain instances, it induces directed chemotaxis in nearby leukocytes (e.g. monocytes). In certain instances, PF4 induces the chemotaxis of a leukocyte to the site of an inflammation or tissue injury.

In certain instances, RANTES and PF4 form a heteromultimer (e.g., a heterodimer). In certain instances, a RANTES and PF4 heteromultimer (e.g., a heterodimer) amplifies the effects of RANTES-induced monocyte arrest. In certain instances, inhibiting the formation of a RANTES/PF4 heteromultimer (e.g., a heterodimer) decreases monocyte arrest.

### Inflammatory Disorders

In some embodiments, the methods and compositions described herein treat inflammation (e.g., acute or chronic). In certain instances, inflammation results from (either partially or fully) an infection. In certain instances, inflammation results from (either partially or fully) damage to a tissue (e.g., by a burn, by frostbite, by exposure to a cytotoxic agent, or by trauma). In certain instances, inflammation results from (either partially or fully) an autoimmune disorder. In certain instances, inflammation results from (either partially or fully) the presence of a foreign body (e.g., a splinter). In certain instances, inflammation results from exposure to a toxin and/or chemical irritant.

As used herein, “acute inflammation” refers to inflammation characterized in that it develops over the course of a few minutes to a few hours, and ceases once the stimulus has been removed (e.g., an infectious agent has been killed by an immune response or administration of a therapeutic agent, a foreign body has been removed by an immune response or extraction, or damaged tissue has healed). The short duration of acute inflammation results from the short half-lives of most inflammatory mediators.

In certain instances, acute inflammation begins with the activation of leukocytes (e.g., monocytes, macrophages, neutrophils, basophils, eosinophils, lymphocytes, dendritic cells, and mastocytes). In certain instances, the leukocytes release inflammatory mediators (e.g., histamines, proteoglycans, serine proteases, eicosanoids, and cytokines). In certain instances, inflammatory mediators result in (either partially or fully) the symptoms associated with inflammation. For example, in certain instances an inflammatory mediator dilates post capillary venules, and increases blood vessel permeability. In certain instances, the increased blood flow that follows vasodilation results in (either partially or fully) rubor and calor. In certain instances, increased permeability of the blood vessels results in an exudation of plasma into the tissue leading to edema. In certain instances, the latter allows leukocytes to migrate along a chemotactic gradient to the site of the inflammatory stimulant. Further, in certain instances, structural changes to blood vessels (e.g., capillaries and venules) occur. In certain instances, the structural changes are induced (either partially or fully) by monocytes and/or macrophages. In certain instances, the structural changes include, but are not limited to, remodeling of vessels, and angiogenesis. In certain instances, angiogenesis contributes to the maintenance of chronic inflammation by allowing for increased transport of leukocytes. Additionally, in certain instances, histamines and bradykinin irritate nerve endings leading to itching and/or pain.

In certain instances, chronic inflammation results from the presence of a persistent stimulant (e.g., persistent acute inflammation, bacterial infection (e.g., by Mycobacterium tuberculosis), prolonged exposure to chemical agents (e.g., silica, or tobacco smoke) and autoimmune reactions (e.g., rheumatoid arthritis)). In certain instances, the persistent stimulant results in continuous inflammation (e.g., due to the continuous recruitment of monocytes, and the proliferation of macrophages). In certain instances, the continuous inflammation further damages tissues which results in the additional recruitment of mononuclear cells thus maintaining and exacerbating the inflammation. In certain instances, physiological responses to inflammation further include angiogenesis and fibrosis.

Multiple disorders are associated with inflammation (i.e., inflammatory disorders). Inflammatory disorders include, but are not limited to, Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, or combinations thereof.

In some embodiments, the methods and compositions described herein treat a T-cell mediated autoimmune disorder. In certain instances, a T-cell mediated autoimmune disorder is characterized by a T-cell mediated immune response against self (e.g., native cells and tissues).

Examples of T-cell mediated autoimmune disorders include, but are not limited to colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, the methods and compositions described herein treat pain. Pain includes, but is not limited to acute pain, acute inflammatory pain, chronic inflammatory pain and neuropathic pain.

In some embodiments, the methods and compositions described herein treat hypersensitivity. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection).

As used herein, “allergy” means a disorder characterized by excessive activation of mast cells and basophils by IgE. In certain instances, the excessive activation of mast cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, the methods and compositions described herein treat angiogenesis. As used herein, “angiogenesis” refers to the formations of new blood vessels. In certain instances, angiogenesis occurs with chronic inflammation. In certain instances, angiogenesis is induced by monocytes and/or macrophages.

In some embodiments the present invention comprises a method of treating a neoplasia. In certain instances, a neoplastic cell induces an inflammatory response. In certain instances, part of the inflammatory response to a neoplastic cell is angiogenesis. In certain instances, angiogenesis facilitates the development of a neoplasia. In some embodiments, the neoplasia is: angiosarcoma, Ewing sarcoma, osteosarcoma, and other sarcomas, breast carcinoma, cecum carcinoma, colon carcinoma, lung carcinoma, ovarian carcinoma, pharyngeal carcinoma, rectosigmoid carcinoma, pancreatic carcinoma, renal carcinoma, endometrial carcinoma, gastric carcinoma, liver carcinoma, head and neck carcinoma, breast carcinoma and other carcinomas, Hodgkins lymphoma and other lymphomas, malignant and other melanomas, parotid tumor, chronic lymphocytic leukemia and other leukemias, astrocytomas, gliomas, hemangiomas, retinoblastoma, neuroblastoma, acoustic neuroma, neurofibroma, trachoma and pyogenic granulomas.

In some embodiments, the methods and compositions described herein treat obesity. As used herein, “obesity” means an accumulation of adipose tissue with a BMI of greater than or equal to 30 kg/m². In certain instances, obesity is characterized a proinflammatory state, increasing the risk of thrombosis. In certain instances, obesity is associated with a low-grade inflammation of white adipose tissue (WAT). In certain instances, WAT associated with obesity is characterized by an increased production and secretion of a wide range of inflammatory molecules including TNF-alpha and interleukin-6 (IL-6). In certain instances, WAT is infiltrated by macrophages, which produce pro-inflammatory cytokines. In certain instances, TNF-alpha is overproduced in adipose tissue. In certain instances, IL-6 production increases during obesity.

In some embodiments, the methods and compositions described herein treat metabolic syndrome. In certain instances, metabolic syndrome is associated with fasting hyperglycemia; high blood pressure; central obesity; decreased HDL levels; elevated triglyceride levels; systemic inflammation; or combinations thereof. In certain instances, metabolic syndrome is characterized by an increase in the levels of C-reactive protein, fibrinogen, (IL-6), and TNFα.

### Anti-Inflammatory Agents

The terms “anti-inflammatory agent” and “modulator of inflammation” are used interchangeably. As used herein, the terms refer to agents treat inflammation and/or an inflammatory disorder. In some embodiments, the anti-inflammatory agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent (e.g., cyclosporine A), an alkylating agent, an anti-metabolite, a plant alkaloid, a terpenoids, a topoisomerase inhibitor, an antitumour antibiotic, an antibody, a hormonal therapy (e.g., aromatase inhibitors), a leukotriene inhibitor, or combinations thereof.

In some embodiments, the second anti-inflammatory agent is: cyclosporine A, alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (Bioinvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AlN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimethyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca),), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), Allcaforsen (ISIS 2302), ATL/TV1102, OGX-011, LY2181308, LY227596, OGX-427, CNT0888, CNTO1275 (ustekinumab) and CNTO148 (golimumab) (both Centocor); MOR103 and MOR202 (Morphosys), Traficet-EN, CCX025, CCX140 and CCX354 (all Chemocentrix), ALN-VSP (Alnylam Pharmaceuticals), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Actos® (Pioglitazone), Avandia® (Rosiglitazone), Amaryl® (Glimepiride), Sulfonylurea-types, Diabeta® (Glyburide), Diabinese® (Chlorpropamide), Glucotrol® (Glipizide), Glynasec (glyburide), Micronase® (glyburide), Orinase® (Tolbutamide), Tolinase® (Tolazamide), Glucophage, Riomet® (Metformin), Glucovance® (glyburide+metformin), Avandamet® (Rosiglitazone+metformin), Avandaryl® (Rosiglitazone+glimepiride), Byetta® (Exenatide), Insulins, Januvia® (Sitagliptin), Metaglip® (glipizide and metformin), Prandin® (Repaglinide), Precose® (Acarbose), Starlix® (Nateglinide), Xenical® (Orlistat), ISIS 113715, OMJP-GCCRRX, OMJP-SGLT2RX, OMJP-GCGRRX, cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AM103 (Amira); AM803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4-yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-05 antibody (e.g., eculizumab); or combinations thereof.

### Cardiovascular Disorders

In some embodiments, the methods and compositions described herein treat a cardiovascular disorder. As used herein, the term “cardiovascular disease” (CVD) refers to a disease or disorder characterized by impairment or dysfunction of the heart, an artery, and/or vein. In some embodiments, the disorder is a dyslipidemia. In some embodiments, the disorder is hyperlipidemia; hypercholesterolemia; hyperglyceridemia; combined hyperlipidemia; hypolipoproteinemia; hypocholesterolemia; abetlipoproteinemia; Tangier disease; or a combination thereof. In some embodiments, the disorder is acute coronary syndrome; unstable angina; non-ST segment elevation myocardial infarction; ST segment elevation myocardial infarction; stable angina; Prinzmetal's angina; arteriosclerosis; atherosclerosis; arteriolosclerosis; stenosis; restenosis; venous thrombosis; arterial thrombosis; stroke; transient ischemic attack; peripheral vascular disease; coronary artery disease; obesity; diabetes; metabolic syndrome; or combinations thereof.

### Lipids and Lipoproteins

In some embodiments, the methods and compositions described herein treat dyslipidemia. As used herein, the term “dyslipidemia” means a disruption (i.e., variation from a normal range) in the concentration of a lipid in the blood.

In certain instances, a dyslipidemia is an increase in lipid (e.g. cholesterol, glycerides, or triglyceride) concentrations over a normal range (i.e., a hyperlipidemia). In certain instances, a hyperlipidemia involves an increase in the concentration of cholesterol (i.e., hypercholesterolemia); glycerides (i.e., hyperglyceridemia); triglycerides (i.e., hypertriglyceridemia); lipoproteins (i.e., hyperlipoproteinemia); chylomicrons (i.e., hyperchylomicronemia); or combinations thereof (e.g., combined hyperlipidemia). In certain instances, a dyslipidemia is a decrease in lipid concentrations below a normal range (i.e., a hypolipidemia). In certain instances, a hypolipidemia involves a decrease in the concentration of lipoproteins (i.e., hypolipoproteinemia); cholesterol (i.e., hypocholesterolemia); beta lipoproteins (i.e., abetalipoproteinemia); HDL (i.e., Tangier disease); or combinations thereof. In certain instances, a dyslipidemia results from environmental factors (e.g., lack of exercise or food intake). In certain instances, a dyslipidemia results from genetic factors (e.g., aberrant expression of ApoA1, Apo B, ApoC2, LPL, or LDL receptor).

In certain instances, blood comprises lipoproteins. In certain instances, a lipoprotein is a complex of proteins (e.g., ApoA1, ApoA2, ApoA4, ApoA5, ApoC1, ApoC2, ApoC3, ApoD, ApoE, LCAT, PAF-AH, PON1, GPX, serum amyloid A, α-1 antitrypsin, and amyloid-β) and lipids. In certain instances, a lipoprotein is a high density lipoprotein (HDL). In certain instances, a lipoprotein is a low density lipoprotein (LDL).

### HDL

HDL is a type of lipoprotein that transports cholesterol and triglycerides to the liver. In certain instances, HDL comprises ApoA1 and ApoA2. In certain instances, ApoA1 and ApoA2 are expressed in the liver. In certain instances, the liver synthesized HDL.

In certain instances, HDL transport cholesterol from cells to the liver, adrenals, ovary and/or testes. In certain instances, cholesterol transported to the liver is excreted as bile. In certain instances, cholesterol transported to adrenals, ovaries and/or testes are used to synthesize steroid hormones.

HDL comprises multiple sub-classes of lipoprotein. In certain instances, the subclasses of HDL differ in size, density, protein and lipid composition. In certain instances, some HDL are protective, anti-oxidative, anti-inflammatory and/or anti-atherogenic. In certain instances, some HDL are neutral. In certain instances, some HDL enhance oxidation, increase inflammation and/or are pro-atherogenic.

In certain instances, increasing the concentration of HDL across all or most sub-classes results in the production of reactive oxygen species (ROS). In certain instances, an enzyme associated with HDL modifies a phospholipid into an oxidized phospholipid. In certain instances, an enzyme associated with HDL modifies cholesterol into an oxidized sterol. In certain instances, an oxidized sterol and/or an oxidized phospholipid results in pro-inflammatory and/or pro-atherogenic HDL.

In certain instances, cholesteryl ester transfer protein (CETP) exchanges triglycerides transported by VLDL (very low density lipoprotein) for cholesteryl esters transported by HDL. In certain instances, the exchange of triglycerides for cholesteryl esters results in VLDL being processed into LDL. In certain instances, LDL is removed from circulation by the LDL receptor pathway. In certain instances, the triglycerides are degraded by hepatic lipase. In certain instances, delipidified HDL recirculate in the blood and transport additional lipids to the liver.

In certain instances, inhibiting CETP disrupts the metabolism of HDL. In certain instances, inhibiting CETP prevents transfer of HDL-cholesterol and increases circulating levels of cholesteryl-ester enriched (larger) HDL subfractions. In some embodiments, inhibiting (partially or fully) CETP treat CVD. In certain instances, slowing the catabolism of HDL increases total circulating HDL levels. In certain instances, increasing total circulating HDL levels treats atherogenesis. In some embodiments, inhibiting (partially or fully) CETP results (partially or fully) in inflammation and/or worsening of CVD. In certain instances, increasing total circulating HDL levels generates a lipid pool with reduced clearance (kinetics). In certain instances, reduced clearance of lipids increases HDL capacity to harbor oxidizable and potentially inflammatory lipid stores.

### LDL

Low-density lipoprotein (LDL) is a type of lipoprotein that transports cholesterol and triglycerides from the liver to peripheral tissues. In certain instances, LDL comprises an apolipoprotein B (ApoB). In certain instances, ApoB is expressed as two isoforms, ApoB48 and ApoB100. In certain instances, ApoB48 is synthesized by intestinal cells. In certain instances, ApoB100 is synthesized in the liver. In certain instances, Hsp110 stabilizes of ApoB.

### Cardiovascular Disorders

In some embodiments, the methods and compositions described herein treat atherosclerosis. As used herein, “atherosclerosis” means inflammation of an arterial wall. In certain instance, the inflammation results from (partially or fully) the accumulation of macrophage white blood cells. In certain instances, the inflammation results from (partially or fully) the presence of oxidized LDL. In certain instances, oxidized LDL damages an arterial wall. In certain instances, monocytes respond to (i.e., follow a chemotactic gradient to) the damaged arterial wall. In certain instances, the monocytes differentiate into macrophages. In certain instances, macrophages endocytose the oxidized-LDL (cells such as macrophages with endocytosed LDL are called “foam cells”). In certain instances, a foam cell dies. In certain instances, the rupture of a foam cell deposits oxidized cholesterol into the artery wall. In certain instances, the arterial wall becomes inflamed due to the damaged caused by the oxidized LDL. In certain instances, cells form a hard covering over the inflamed area. In certain instances, the cellular covering narrows an artery.

In certain instances, an atheromatous plaque is divided into three distinct components: (a) the atheroma (i.e., a nodular accumulation of a soft, flaky, yellowish material comprised of macrophages nearest the lumen of the artery; (b) areas of cholesterol crystals; and (c) calcification at the outer base.

In certain instances, an atherosclerotic plaque results (partially or fully) in stenosis (i.e., the narrowing of blood vessel). In certain instances, stenosis results (partially or fully) in decreased blood flow. In some embodiments, the methods and compositions described herein treat stenosis and/or restenosis. In certain instances, an atherosclerotic plaque results (partially or fully) in the development of an aneurysm. In some embodiments, the methods and compositions described herein treat an aneurysm. In certain instances, the rupture of an atherosclerotic plaque results (partially or fully) in an infarction (i.e., the deprivation of oxygen) to a tissue. In some embodiments, the methods and compositions described herein treat an infarction.

In some embodiments, the methods and compositions described herein treat a myocardial infarction. “Myocardial infarction” and “heart attack” are used interchangeably. As used herein, both terms refer to an interruption in the blood supply to the heart. In certain instances, an interruption in the blood supply to the heart results from (partially or fully) the occlusion of a coronary artery by a ruptured atherosclerotic plaque. In certain instances, occlusion of an artery results in the infarction of myocardium. In certain instances, the infarction of myocardium results in the scarring of myocardial tissue. In certain instances, scarred of myocardial tissue conducts electrical impulses more slowly than unscarred tissue. In certain instances, the difference in conduction velocity between scarred and unscarred tissue results (partially or fully) in ventricular fibrillation or ventricular tachycardia.

In some embodiments, the methods and compositions described herein treat an angina (e.g., stable or unstable). As used herein, “angina pectoris” refers chest pain resulting from (partially or fully) of the heart.

In some embodiments, the methods and compositions described herein treat a thrombosis (venous or arterial). As used herein, “thrombosis” refers to the formation of a blood clot. In certain instances, the blood clot forms in a vein (i.e., venous thrombosis). In certain instances, the blood clot forms in an artery (i.e., arterial thrombosis). In certain instances, a piece of or the entire blood clot is transported (i.e., an embolism) to the lungs (i.e., a pulmonary embolism). In some embodiments, the methods and compositions described herein treat an embolism.

In some embodiments, the methods and compositions described herein treat a stroke. As used herein, “stroke” refers to a loss of brain function (e.g., necrosis of brain tissue) resulting from (partially or fully) a disturbance in blood supply (e.g., ischemia). In certain instances, a stroke results from (partially or fully) a thrombosis or an embolism.

In certain instances, an atherosclerotic plaque results (partially or fully) in the development of an aneurysm. In some embodiments, the methods and compositions described herein treat an aneurysm. In some embodiments, the methods and compositions described herein treat an abdominal aortic aneurysm (“AAA”). As used herein, an “abdominal aortic aneurysm” is a localized dilatation of the abdominal aorta. In certain instances, the rupture of an AAA results in bleeding, leading to hypovolemic shock with hypotension, tachycardia, cyanosis, and altered mental status.

In some embodiments, the compositions and methods disclosed herein treat abdominal aortic aneurysms. In certain instances, abdominal aortic aneurysms result (partially or fully) from an extensive breakdown of structural proteins (e.g., elastin and collagen). In some embodiments, a method and/or composition disclosed herein partially or fully inhibits the breakdown of a structural protein (e.g., elastin and collagen). In certain instances, the breakdown of structural proteins is caused by activated MMPs. In some embodiments, a method and/or composition disclosed herein partially or fully inhibits the activation of an MMP. In some embodiments, a composition and/or method disclosed herein inhibit the upregulation of MMP-1, MMP-9 or MMP-12. In certain instances, MIF is co-expressed with MMP-1, MMP-9, and MMP-12 in abdominal aortic aneurysms. In certain instances, the MIF is upregulated in stable abdominal aortic aneurysm and is intensified further in ruptured aneurysms. In certain instances, MMPs are activated following infiltration of a section of the abdominal aorta by leukocytes (e.g., macrophages and neutrophils). In some embodiments, a method and/or composition disclosed herein partially or fully inhibits the activity of MIF. In some embodiments, a method and/or composition disclosed herein partially or fully inhibits the infiltration of a section of the abdominal aorta by leukocytes.

### Treatments for Cardiovascular Disorders

In some embodiments, the cardiovascular disorder is treated with an active agent (the “cardiovascular disorder agent”). In some embodiments, the active agent is niacin; a fibrate; a statin; an apolipoprotein A-1 modulator; an ACAT modulator; a CETP modulator; a glycoprotein IIb/IIIa modulator; a P2Y12 modulator; an Lp-PLA2 modulator; or combinations thereof.

In some embodiments, the cardiovascular disorder agent reduces the risk of developing a cardiovascular disorder across all levels of HDL. In some embodiments, the cardiovascular disorder agent inhibits (partially or fully) the activity of 3-hydroxy-3-methylglutaryl coenzyme A reductase. In some embodiments, the cardiovascular disorder agent is a atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof.

In some embodiments, the cardiovascular disorder agent raises HDL non-selectively. In some embodiments, the cardiovascular disorder agent down-regulates transcription of a CETP gene. In some embodiments, the cardiovascular disorder agent is niacin.

In some embodiments, the cardiovascular disorder agent reduces the risk of developing a cardiovascular disorder in individuals with low HDL with metabolic syndrome. In some embodiments, the cardiovascular disorder agent is bezafibrate; ciprofibrate; clofibrate; gemfibrozil; fenofibrate; or combinations thereof.

In some embodiments, the cardiovascular disorder agent selectively increases the levels of apoA1 protein (e.g. by transcriptional induction of the gene encoding apoA1) and increases the production of nascent HDL (apoA1-enriched). In some embodiments, the second active agent is DF4 (Ac-D-W-F-K-A-F-Y-D-K-V-A-E-K-F-K-E-A-F-NH2); DF5; RVX-208 (Resverlogix); or combinations thereof.

In some embodiments, the cardiovascular disorder agent inhibits the activity of Acyl-CoA cholesteryl acyl transferase (ACAT). In some embodiments, the cardiovascular disorder agent inhibits (partially or fully) the formation of foam cells and the accumulation of cholesterol esters in macrophages and vascular tissue. In some embodiments, the second active agent is avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof.

In some embodiments, the cardiovascular disorder agent inhibits (partially or completely) the activity of Cholesteryl Ester Transfer Protein (CETP). In some embodiments, the cardiovascular disorder agent increases HDL-C concentration and reduces LDL-C concentration. In some embodiments, the cardiovascular disorder agent increases antioxidant enzymes associated with HDL and decreases oxidized LDL. In some embodiments, the cardiovascular disorder agent is torcetrapib; anacetrapid; JTT-705 (Japan Tobacco/Roche); or combinations thereof.

In some embodiments, the cardiovascular disorder agent inhibits (partially or fully) the activity of glycoprotein IIb/IIIa. In some embodiments, the cardiovascular disorder agent prevents (partially or fully) platelet aggregation and/or thrombus formation. In some embodiments, the cardiovascular disorder agent is abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1-[3-(piperidin-4-yl) propionyl]piperidin-3-ylcarbonyl]amino]propionic acid trihydrate); or combinations thereof.

In some embodiments, the cardiovascular disorder agent antagonizes P2Y12. In some embodiments, the cardiovascular disorder agent inhibits (partially or fully) platelet aggregation. In some embodiments, the cardiovascular disorder agent is clopidogrel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences) or combinations thereof.

In some embodiments, the cardiovascular disorder agent inhibits (partially or fully) the activity of lipoprotein-associated phospholipase A2 (lp-PLA2). In some embodiments, the cardiovascular disorder agent inhibits (partially of fully) the hydrolysis of the center (sn-2) ester bond of phospholipids. In some embodiments, the cardiovascular disorder agent inhibits (partially or fully) the production of oxidized fatty acids and lysophosphatidyl choline. In some embodiments, the cardiovascular disorder agent inhibits (partially or fully) the chemotaxis of monocytes. In some embodiments, the cardiovascular disorder agent is darapladib (SB 480848); SB-435495 (GlaxoSmithKline); SB-222657 (GlaxoSmithKline); SB-253514 (GlaxoSmithKline); or combinations thereof.

In some embodiments, the cardiovascular disorder agent inhibits a leukotriene (e.g., by antagonizing LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; or by inhibiting the synthesis of a leukotriene via 5-LO, FLAP, LTA4H, LTA4S, or LTC4S). In some embodiments, the second active agent is an antagonist of 5-LO. In some embodiments, the second active agent is an antagonist of FLAP. In some embodiments, the second active agent is A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AM103 (Amira); AM803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4-yl)phenoxy)methyl)-1-methyl-2(1H)-quinlolinone); or combinations thereof.

In some embodiments, the cardiovascular disorder agent is administered before, after, or simultaneously with the modulator of inflammation.

In some embodiments, a cardiovascular disorder is treated by delipidifying the blood of an individual. In some embodiments, the blood of an individual is delipidified by removing a lipid from an HDL molecule in an individual in need thereof. In some embodiments, administering a therapeutically-effective amount of a modulator of inflammation acts in synergy with the removal of a lipid from an HDL molecule.

### Small Molecule Antagonists of RANTES and PF4

In some embodiments, the formation of a RANTES/PF4 heteromultimer (e.g., a heterodimer) is disrupted by use of a small molecule that binds to RANTES and/or a small molecule that binds to PF4. In some embodiments, the small molecule antagonizes or inhibits (both partially or completely) the interaction of PF4 and RANTES.

In some embodiments, the function of a RANTES/PF4 heteromultimer (e.g., a heterodimer) is disrupted by use of a small molecule that binds to a RANTES/PF4 heterodimer.

### Antibody Antagonists of RANTES and PF4

In some embodiments, the formation of a RANTES/PF4 heteromultimer (e.g., a heterodimer) is disrupted by use of an antibody that binds to RANTES and/or an antibody that binds to PF4. In some embodiments, the antibody antagonizes or inhibits (both partially or completely) the interaction of PF4 and RANTES.

In some embodiments, the function of a RANTES/PF4 heteromultimer (e.g., a heterodimer) is disrupted by use of an antibody that binds to a RANTES/PF4 heterodimer.

The antibodies herein include monoclonal, polyclonal, recombinant, chimeric, humanized, bi-specific, grafted, human, and fragments thereof including antibodies altered by any means to be less immunogenic in humans. Thus, for example, the monoclonal antibodies and fragments, etc., herein include “chimeric” antibodies and “humanized” antibodies. In general, chimeric antibodies include a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity. For example in some embodiments a chimeric antibody contains variable regions derived from a mouse and constant regions derived from human in which the constant region contains sequences homologous to both human IgG2 and human IgG4.

“Humanized” forms of non-human (e.g., murine) antibodies or fragments are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include, grafted antibodies or CDR grafted antibodies wherein part or all of the amino acid sequence of one or more complementarity determining regions (CDRs) derived from a non-human animal antibody is grafted to an appropriate position of a human antibody while maintaining the desired binding specificity and/or affinity of the original non-human antibody. In some embodiments, corresponding non-human residues replace Fv framework residues of the human immunoglobulin. In some embodiments humanized antibodies comprise residues that are found neither in the recipient antibody nor in the imported CDR or framework sequences. These modifications are made to further refine and optimize antibody performance. In some embodiments, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence.

### Peptide Antagonists of RANTES and PF4

In some embodiments, the interaction of RANTES and PF4 is disrupted by use of a peptide antagonist that mimics all or part of RANTES. In some embodiments, the interaction of RANTES and PF4 is disrupted by use of a peptide antagonist that mimics the PF4 interacting domain of RANTES. In certain instances, PF4 binds to the peptide antagonist and thus does not bind to RANTES.

In some embodiments, the peptide antagonist is an isolated peptide, pharmacologically acceptable salts, derivatives, and conjugates thereof. In some embodiments, the peptide antagonist comprises a portion of a RANTES amino acid sequence.

In some embodiments, the peptide antagonists described herein do not effect (or only partially effect) the other functions of the RANTES and/or PF4. In one embodiment, a selective blocking of the recruiting of monocytes is achieved, for example, on endothelium.

In some embodiments, the peptide antagonists described herein provide a high specificity, and do not effect (or only partially effect) the many metabolic processes mediated by the chemokines RANTES and PF4, for example, the immune or clotting systems.

In some embodiments, peptide antagonists comprises between 15 and 25 amino acids. In some embodiments, peptide antagonists comprise between 19 and 25 amino acids. In some embodiments, a peptide antagonist described herein has a length of no more than 25 amino acids. In a further embodiment, the peptide antagonist has a number of amino acids in the range of about 15 to about 25 amino acids, and in a further embodiment, in the range of about 15 to about 22 amino acids. In further embodiments, the peptide antagonist has a number of amino acids in the range of about 18 to about 23 amino acids, including in the range of about 18 to about 22 amino acids, and including, in the range of about 19 to about 22 amino acids, and also including in the range of about 20 to about 21 amino acids. In certain embodiments, the peptide has 22 amino acids.

In one embodiment, the peptide antagonists described herein have a cysteine residue at each of the amino-terminal and carboxy-terminal ends. In some embodiments, the cysteine residue at the amino-terminus and the cysteine residue at the carboxy terminus bind together, yielding a ring. In one embodiment, a cyclical peptide antagonist has an improved stability. In one embodiment, the peptide antagonists described herein have a longer effectiveness and, accordingly, are used in smaller amounts.

In some embodiments, the peptide antagonists described herein are prepared by any suitable manner (e.g., literature methods).

In some embodiments, the peptide antagonist has an amino acid sequence SEQ ID NO: 1, as indicated below:

C-X1-X2-YFYTS-X3-X4-X5-X6-X7-X8-X9-X10-X11-X12-
X13-X14-X15-C,

wherein
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine;
X15 is chosen from the group containing arginine, lysine, alanine, glutamine, histidine and asparagine, or an amino acid deletion.

In some embodiments, the peptide antagonist is derived from a human RANTES amino acid sequence. In certain instances, human RANTES is encoded by a nucleotide sequence located on chromosome 17 at the cytogenic band 17q12 (per Ensemble cytogenic band) or 17q11.2-q12 (per Entrez Gene). In some embodiments, the peptide antagonist comprises a portion of a human RANTES amino acid sequence. In some embodiments, the peptide antagonist has an amino acid sequence SEQ ID NO: 2, as indicated below:

C-KEYFYTSGKCSNPAVVFVTR-C.

In some embodiments, the peptide antagonist is derived from a mouse RANTES amino acid sequence. In certain instances, mouse RANTES is encoded by a nucleotide sequence located on chromosome 11 at the locus 11 (47.40 cM)₄. In some embodiments, the peptide antagonist comprises a portion of a mouse RANTES amino acid sequence. In some embodiments, the peptide antagonist has an amino acid sequence SEQ ID NO: 3, as indicated below:

C-KEYFYTSSKCSNLAVVFVTR-C.

In some embodiments, the peptide antagonist is derived from a pig RANTES amino acid sequence. In some embodiments, the peptide antagonist comprises a portion of a pig RANTES amino acid sequence. In some embodiments, the peptide antagonist has an amino acid sequence SEQ ID NO: 4, as indicated below:

C-QEYFYTSSKCSMAAVVFITR-C.

In some embodiments, the peptide antagonist has an amino acid sequence SEQ ID NO 5, as indicated below:

C-X1-X2-YFYTS-X3-X4-X5-X6-X7-X8-X9-X10-X11-X12-X13-X14-X15-C;

where:
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, lysine, alanine, glutamine, histidine and asparagine, or an amino acid deletion.

In some embodiments, the peptide antagonist has an amino acid sequence SEQ ID NO: 6, as indicated below:

(SEQ ID NO: 6)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion; X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing serine, glycine and threonine;
X7 is chosen from the group containing methionine, isoleucine, leucine, and phenylalanine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 7, as indicated below:

(SEQ ID NO: 7)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing serine, glycine and threonine;
X7 is chosen from the group containing asparagine and glutamine;
X8 is chosen from the group containing leucine, isoleucine, phenylalanine, alanine, valine, threonine and methionine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 8, as indicated below:

(SEQ ID NO: 8)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing methionine, isoleucine, leucine, and phenylalanine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 9, as indicated below:

(SEQ ID NO: 9)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing serine, glycine and threonine;
X7 is chosen from the group containing methionine, isoleucine, leucine, and phenylalanine;
X8 is chosen from the group containing leucine, isoleucine, phenylalanine, alanine, valine, threonine and methionine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 10, as indicated below:

(SEQ ID NO: 10)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing leucine, isoleucine, phenylalanine, alanine, valine, threonine and methionine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 11, as indicated below:

(SEQ ID NO: 11)
C-X1-X2-YFYTS-X3-X4-X5-X6-X7-X8-X9-X10-X11-X12-X13-X14-X15-C

where:
X1 is chosen from the group containing lysine, glutamine, arginine, histidine and asparagine, or an amino acid deletion;
X2 is chosen from the group containing glutaminic acid, asparaginic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing methionine, isoleucine, leucine, and phenylalanine;
X8 is chosen from the group containing leucine, isoleucine, phenylalanine, alanine, valine, threonine and methionine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine; and
X15 is chosen from the group containing arginine, alanine, lysine, glutamine, histidine and asparagine, or an amino acid deletion.

In some embodiments, the peptide antagonist has an amino acid sequence SEQ ID NO: 12, as indicated below:

(SEQ ID NO: 12)
C-X1-X2-YFYTS-X3-X4-X5-X6-X7-X8-X9-X10-X11-X12-X13-X14-X15-C

where:
X1 is chosen from the group containing lysine, glutamine, arginine, histidine and/or asparagine, or an amino acid deletion;
X2 is chosen from the group containing glutaminic acid, asparaginic acid and/or glutamine, or an amino acid deletion;
X3 is chosen from the group containing glycine, serine and/or alanine;
X4 is chosen from the group containing lysine, leucine and/or arginine;
X5 is chosen from the group containing serine, cysteine, glycine and/or threonine;
X6 is chosen from the group containing serine, glycine and/or threonine;
X7 is chosen from the group containing asparagine and/or glutamine;
X8 is chosen from the group containing proline, tyrosine and/or glycine;
X9 is chosen from the group containing glycine, alanine and/or serine;
X10 is chosen from the group containing isoleucine, valine and/or asparagine;
X11 is chosen from the group containing valine, isoleucine and/or asparagine;
X12 is chosen from the group containing phenylalanine, tyrosine, isoleucine, valine, leucine and/or methionine;
X13 is chosen from the group containing isoleucine, valine, leucine, methionine and/or phenylalanine;
X14 is chosen from the group containing threonine, glycine, alanine, serine and/or tyrosine; and
X15 is chosen from the group containing arginine, lysine, glutamine, histidine and/or asparagine, or an amino acid deletion.

In some embodiments, the peptide antagonist has an amino acid sequence SEQ ID NO: 13, as indicated below:

(SEQ ID NO: 13)
C-KEYFYTSSKSSNLAVVFVTR-C.

In some embodiments, the peptide antagonist has an amino acid sequence SEQ ID NO: 14, as indicated below:

(SEQ ID NO 14)
C-SFKGTTVYALSNVRSYSFVK-C.

In some embodiments, the peptide antagonist has an amino acid sequence SEQ ID NO: 15, as indicated below:

(SEQ ID NO 15)
C-SFKGTNVYALTKVRSYSFVS-C.

In some embodiments, the peptide antagonist has any amino acid sequence list in Table 1.

TABLE 1

| Species | Sequence |
|---|---|
| Homo sapien | KEYFYTSGKCSNPAVVFVTR |
| | (SEQ ID NO 16) |
| Pan troglodytes | KEYFYTSGKCSNPAVV |
| | (SEQ ID NO 17) |
| Pongo pygmaeus | KEYFYTSGKCSNPAVVFVTR |
| | (SEQ ID NO 18) |
| Macaca mulatta | KEYFYTSGKCSNPAVVFVTR |
| | (SEQ ID NO 19) |
| Otolemur | KEYFYTSGKCSNPAVVFITR |
| garnettii | (SEQ ID NO 20) |
| Microcebus | MEYFYTSGKCSNPAVVFITR |
| murinus | (SEQ ID NO 21) |
| Ochotona | KEYFYTSGKCSNPAVVFVTR |
| princeps | (SEQ ID NO 22) |
| Oryctolagus | TEYFYTSGKCSFPAVVFVTR |
| cuniculus | (SEQ ID NO 23) |
| Mus musculus | KEYFYTSSKCSNLAVVFVTR |
| | (SEQ ID NO 24) |
| Rattus | KEYFYTSSKCSNLAVVFVTR |
| norvegicus | (SEQ ID NO 25) |
| Peromyscus | KEYFYTSSKCSNSAVVFVTR |
| maniculatus | (SEQ ID NO 26) |
| Sigmodon | KEYFYTSSKCSNFAVVFVTR |
| hispidus | (SEQ ID NO 27) |
| Cavia porcellus | KEYFYTSSKCSNLAVVFVTR |
| | (SEQ ID NO 28) |
| Spermophilus | KEYFYTSSKCSNLAV |
| tridecemlineatus | (SEQ ID NO 29) |
| Felis catus | QEYFYTSSKCSMPAVVFVTR |
| | (SEQ ID NO 30) |
| Canis lupus | QEYFYTSSKCSMPAVVFVTR |
| familiaris | (SEQ ID NO 31) |
| Sus scrofa | QEYFYTSSKCSMAAVVFITR |
| | (SEQ ID NO 32) |
| Bos taurus | QEYFYTSSKCSMAAVVFITR |
| | (SEQ ID NO 33) |
| Equus caballus | QEYFYTSSKCSIPAVVFVTR |
| | (SEQ ID NO 34) |
| Monodelphis | REYFYTSSRCGNLGVVFITR |
| domestica | (SEQ ID NO 35) |
| Loxodonta | KEYFYTSGKCSMPAV |
| africana | (SEQ ID NO 36) |
| Dasypus | KEYFYTSGKCSNPAV |
| novemcinctus | (SEQ ID NO 37) |
| Echinops | REYFYTSSKCTSPAVVFVTR |
| telfairi | (SEQ ID NO 38) |
| Erinaceus | QEYFYTSSKCSIPSAVVFVTR |
| europaeus | (SEQ ID NO 39) |
| Tupaia | REYFYTSGKCSNPAVVFITR |
| belangeri | (SEQ ID NO 40) |
| Sorex araneus | QDYFYTSSKCSMPAVVFVTR |
| | (SEQ ID NO 41) |
| Gallus gallus | KDYFYTSSKCPQAAVVFITR |
| | (SEQ ID NO 42) |
| Anas | KDYFYTSSKCPQPAVVFITR |
| platyrhynchos | (SEQ ID NO 43) |
| Myotis | QEYFYTSSKCSMPAVVLITR |
| lucifugus | (SEQ ID NO 44) |

### Metabolites

In some embodiments, the antagonist of PF4/RANTES interaction is a fragment of any peptide sequence disclosed herein (hereinafter, “peptide fragment”). As used herein, “peptide fragment” means an amino acid polymer produced by cleaving any peptide of SEQ ID NO 1 through SEQ ID NO 44. In some embodiments, a peptide of SEQ ID NO 1 through SEQ ID NO 44 is cleaved at one site (e.g., one peptide bond is broken). In some embodiments, a peptide of SEQ ID NO 1 through SEQ ID NO 44 is cleaved at two sites (e.g., two peptide bonds are broken). In some embodiments, the peptide fragment is produced by the metabolism of any peptide of SEQ ID NO 1 through SEQ ID NO 44.

In some embodiments, the fragment has structural features similar to a peptide disclosed herein. In some embodiments, the fragment is linear.

In some embodiments, the fragment has between 5 and 10 amino acids. In some embodiments, the fragment has 5 amino acids. In some embodiments, the fragment has between 6 and 10 amino acids. In some embodiments, the fragment has 6 amino acids. In some embodiments, the fragment has between 7 and 10 amino acids. In some embodiments, the fragment has between 8 and 10 amino acids. In some embodiments, the fragment has between 9 and 10 amino acids.

In some embodiments, the metabolite has a formula selected from:

```
C-X1-X2-X3-X4-X5-T-X6-X7-X8-X9-S-N-X10-X11-X12-X13-X14-X15-X16
C-X1-X2-X3-X4-X5-T-X6-X7-X8-X9-S-N-X10-X11-X12-X13-X14-X15
C-X1-X2-X3-X4-X5-T-X6-X7-X8-X9-S-N-X10-X11-X12-X13-X14
C-X1-X2-X3-X4-X5-T-X6-X7-X8-X9-S-N-X10-X11-X12-X13
C-X1-X2-X3-X4-X5-T-X6-X7-X8-X9-S-N-X10-X11-X12
C-X1-X2-X3-X4-X5-T-X6-X7-X8-X9-S-N-X10-X11
C-X1-X2-X3-X4-X5-T-X6-X7-X8-X9-S-N-X10
C-X1-X2-X3-X4-X5-T-X6-X7-X8-X9-S-N
C-X1-X2-X3-X4-X5-T-X6-X7-X8-X9
C-X1-X2-X3-X4-X5-T-X6-X7-X8
C-X1-X2-X3-X4-X5-T-X6-X7
C-X1-X2-X3-X4-X5-T-X6
C-X1-X2-X3-X4-X5-T
X1-X2-X3-X4-X5-T-X6-X7-X8-X9-S-N-X10-X11-X12-X13-X14-X15-X16-K-C
  X2-X3-X4-X5-T-X6-X7-X8-X9-S-N-X10-X11-X12-X13-X14-X15-X16-K-C
    X3-X4-X5-T-X6-X7-X8-X9-S-N-X10-X11-X12-X13-X14-X15-X16-K-C
      X4-X5-T-X6-X7-X8-X9-S-N-X10-X11-X12-X13-X14-X15-X16-K-C
        X5-T-X6-X7-X8-X9-S-N-X10-X11-X12-X13-X14-X15-X16-K-C
          T-X6-X7-X8-X9-S-N-X10-X11-X12-X13-X14-X15-X16-K-C
            X6-X7-X8-X9-S-N-X10-X11-X12-X13-X14-X15-X16-K-C
              X7-X8-X9-S-N-X10-X11-X12-X13-X14-X15-X16-K-C
                X8-X9-S-N-X10-X11-X12-X13-X14-X15-X16-K-C
                  X9-S-N-X10-X11-X12-X13-X14-X15-X16-K-C
                    S-N-X10-X11-X12-X13-X14-X15-X16-K-C
                      N-X10-X11-X12-X13-X14-X15-X16-K-C
C-X1-X2-X3-X4-X5-T-X6-X7-X8-X9-S-N
C-X1-X2-X3-X4-X5-T-X6-X7-X8-X9-S
C-X1-X2-X3-X4-X5-T-X6-X7-X8-X9
C-X1-X2-X3-X4-X5-T-X6-X7-X8
C-X1-X2-X3-X4-X5-T-X6-X7
C-X1-X2-X3-X4-X5-T-X6
C-X1-X2-X3-X4-X5-T
T-6-X7-X8-X9-S-N-X10-X11-X12-X13-X14-X15-X16-K-C
  X6-X7-X8-X9-S-N-X10-X11-X12-X13-X14-X15-X16-K-C
    X7-X8-X9-S-N-X10-X11-X12-X13-X14-X15-X16-K-C
        X8-X9-S-N-X10-X11-X12-X13-X14-X15-X16-K-C
          X9-S-N-X10-X11-X12-X13-X14-X15-X16-K-C
            S-N-X10-X11-X12-X13-X14-X15-X16-K-C
              N-X10-X11-X12-X13-X14-X15-X16-K-C
```

wherein
X1 is selected from serine and lysine;
X2 is selected from glutamic acid, phenylalanine and serine;
X3 is selected from lysine and tyrosine;
X4 is selected from phenylalanine and glycine;
X5 is selected from threonine and tyrosine;
X6 is selected from serine and valine;
X7 is selected from serine and tyrosine;
X8 is selected from alanine and lysine;
X9 is selected from leucine and serine;
X10 is selected from leucine and valine;
X11 is selected from alanine and arginine;
X12 is selected from serine and valine;
X13 is selected from valine and tyrosine;
X14 is selected from phenylalanine and serine;
X15 is selected from phenylalanine and valine; and
X16 is selected from threonine and valine.

In some embodiments, the antagonist of PF4/RANTES interaction is: SSKSSNLAVVFVTRCCKEYFYT (SEQ ID NO 45); SKSSNLAVVFVTRCCKEYFYTS (SEQ ID NO 46); KSSNLAVVFVTRCCKEYFYTSS (SEQ ID NO 47); SSNLAVVFVTRCCKEYFYTSSK (SEQ ID NO 48); SNLAVVFVTRCCKEYFYTSSKS (SEQ ID NO 49); NLAVVFVTRCCKEYFYTSSKSS (SEQ ID NO 50); or a combination thereof. In some embodiments, the antagonist of PF4/RANTES interaction is: SFKGTTVYALSNVRSYSFVKCC (SEQ ID NO 51); FKGTTVYALSNVRSYSFVKCCS (SEQ ID NO 52); SNVRSYSFVKCCSFKGTTVYAL (SEQ ID NO 53); NVRSYSFVKCCSFKGTTVYALS (SEQ ID NO 54); SYSFVKCCSFKGTTVYALSNVR (SEQ ID NO 55); YSFVKCCSFKGTTVYALSNVRS (SEQ ID NO 56); SFVKCCSFKGTTVYALSNVRSY (SEQ ID NO 57); FVKCCSFKGTTVYALSNVRSYS (SEQ ID NO 58); or a combination thereof.

### SAR Chemistry

In some embodiments, any of the aforementioned peptides and/or peptide fragments is used as a “model” to do structure-activity relationship (SAR) chemistry. In some embodiments, the SAR chemistry yields smaller peptides. In some embodiments, the smaller peptides yield small molecules that disrupt the activity of RANTES and/or PF4 (e.g., by figuring out the amino acid residues involved in disrupting the activity of RANTES and/or PF4).

### Peptide Mimetics

In some embodiments, a peptide mimetic is used in place of the peptides described herein, including for use in the treatment or prevention of the diseases disclosed herein.

Peptide mimetics (and peptide-based inhibitors) are developed using, for example, computerized molecular modeling. Peptide mimetics are designed to include structures having one or more peptide linkages optionally replaced by a linkage selected from the group consisting of: —CH₂NH—, —CH₂S—, —CH₂—CH₂—, —CH═CH-(cis and trans), —CHF-(trans), —CoCH₂—, —CH(OH)CH₂—, and —CH₂SO—. In some embodiments such peptide mimetics have greater chemical stability, enhanced pharmacological properties (half-life, absorption, potency, efficacy, etc.), altered specificity (e.g., a broad-spectrum of biological activities), reduced antigenicity, and are more economically prepared. In some embodiments peptide mimetics include covalent attachment of one or more labels or conjugates, directly or through a spacer (e.g., an amide group), to non-interfering positions(s) on the analog that are predicted by quantitative structure-activity data and/or molecular modeling. Such non-interfering positions generally are positions that do not form direct contacts with the receptor(s) to which the peptide mimetic binds to produce the therapeutic effect. In some embodiments, systematic substitution of one or more amino acids of a consensus sequence with a D-amino acid of the same type (e.g., D-lysine in place of L-lysine) are used to generate more stable peptides with desired properties.

In some embodiments, a peptide mimetic is generated by use of a phage display peptide libraries. For disclosure regarding the creation of a phage display peptide library see Scott, J. K. et al. (1990) Science 249:386; Devlin, J. J. et al. (1990) Science 249:404; U.S. Pat. No. 5,223,409, U.S. Pat. No. 5,733,731; U.S. Pat. No. 5,498,530; U.S. Pat. No. 5,432,018;U.S. Pat. No. 5,338,665;U.S. Pat. No. 5,922,545; WO 96/40987 and WO 98/15833 each of which is incorporated by reference for such disclosure. In such libraries, random peptide sequences are displayed by fusion with coat proteins of filamentous phage. Typically, the displayed peptides are affinity-eluted against an antibody-immobilized extracellular domain (in this case PF4 or RANTES. In some embodiments peptide mimetics are isolated by biopanning. In some embodiments whole cells expressing PF4 or RANTES are used to screen the library utilizing FACs to isolate phage bound cells. The retained phages are enriched by successive rounds of biopanning and repropagation. The best binding peptides are sequenced to identify key residues within one or more structurally related families of peptides. The peptide sequences also suggest which residues to replace by alanine scanning or by mutagenesis at the DNA level. In some embodiments mutagenesis libraries are created and screened to further optimize the sequence of the best binders.

In some embodiments structural analysis of protein-protein interaction is used to suggest peptides that mimic the binding activity of the peptides described herein. In some embodiments the crystal structure resulting from such an analysis suggests the identity and relative orientation of critical residues of the peptide, from which a peptide is designed.

For further disclosure re PF4/RANTES, methods of treatment comprising inhibiting the interactions between PF4 and RANTES, and pharmaceutical compositions comprising PF4 and RANTES antagonists see U.S. Provisional Application 61/103,1872, filed Oct. 6, 2008; and PCT International Publication No. WO 2007/042263, which are incorporated by reference herein for such disclosures.

### Combinations

Disclosed herein, in certain embodiments, are methods and pharmaceutical compositions for modulating an inflammatory disorder comprising co-administering (a) a therapeutically-effective amount of a first active agent that inhibits the interaction between RANTES and Platelet Factor 4; and (b) a therapeutically-effective amount of a second active agent selected from an agent that treats an inflammatory disorder through an alternative pathway.

In some embodiments, combining (a) the first active agent; and (b) the second active agent is synergistic and results in a more efficacious therapy. In some embodiments, the therapy is more efficacious as it treats inflammatory disorders by multiple pathways. In some embodiments, the therapy is more efficacious as it treats inflammatory disorders by multiple pathways and treats and/or ameliorates undesired inflammation resulting from the second agent. In some embodiments, the therapy is more efficacious as it allows (partially or fully) a medical professional to increase the prescribed dosage of the second active agent.

### General Inflammatory Disorders

In some embodiments, the first active agent (i.e., a MIF antagonist and/or a modulator of an interaction between RANTES and Platelet Factor 4), and a second anti-inflammatory agent, (e.g., an immunosuppressant) synergistically treat an inflammatory disorder by (1) decreasing the chemotaxis of leukocytes, and (2) reducing the influx of cytokines.

In some embodiments, the second anti-inflammatory agent is: cyclosporine A, alefacept, efalizumab, methotrexate, acitretin, isotretinoin, hydroxyurea, mycophenolate mofetil (MMF), sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (Bioinvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AlN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca),), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Actos® (Pioglitazone), Avandia® (Rosiglitazone), Amaryl® (Glimepiride), Sulfonylurea-types, Diabeta® (Glyburide), Diabinese® (Chlorpropamide), Glucotrol® (Glipizide), Glynasec (glyburide), Micronase® (glyburide), Orinase® (Tolbutamide), Tolinase® (Tolazamide), Glucophage, Riomet® (Metformin), Glucovance® (glyburide+metformin), Avandamet® (Rosiglitazone+metformin), Avandaryl® (Rosiglitazone+glimepiride), Byetta® (Exenatide), Insulins, Januvia® (Sitagliptin), Metaglip® (glipizide and metformin), Prandin® (Repaglinide), Precose® (Acarbose), Starlix® (Nateglinide), Xenical® (Orlistat), cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AM103 (Amira); AM803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4-yl)phenoxy)methyl)-1-methyl-2(1H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-05 antibody (e.g., eculizumab); or combinations thereof.

In certain instances, administration of a 5-ASA causes (either partially or fully) inflammation. In certain instances, administration of sulfasalazine results in (either partially or fully) pneumonitis with or without eosinophilia, vasculitis, pericarditis with or without tamponade, hepatitis, allergic myocarditis, pancreatitis, nephritis, exfoliative dermatitis, serum vasculitis, and/or pleuritis. In certain instances, administration of mesalamine results in (either partially or fully) pericarditis, myocarditis, pancreatitis, hepatitis, interstitial pneumonitis, pleuritis, interstitial nephritis, and/or pneumonitis. In certain instances, administration of olsalazine results in (either partially or fully) myocarditis, pericarditis, pancreatitis, interstitial and/or nephritis.

In some embodiments, the first active agent and a 5-ASA treat an inflammatory disorder by (1) decreasing the chemotaxis of leukocytes, and (2) reducing the synthesis of eicosanoids and inflammatory cytokines. In some embodiments, the first active agent also decreases any undesired inflammation (e.g., pancreatitis) resulting from administration of the 5-ASA.

In some embodiments, the first active agent and an anti-TNF agent treat an inflammatory disorder by (1) decreasing the chemotaxis of leukocytes, and (2) suppressing a TNF-induced cytokine cascade. In some embodiments, the first active agent also decreases any undesired inflammation (e.g., tuberculosis) resulting from administration of the anti-TNF agent.

In some embodiments, the first active and a leukotriene inhibitor treat an inflammatory disorder by (1) decreasing the chemotaxis of leukocytes, and (2) antagonizing LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; or inhibiting the synthesis of a leukotriene via 5-LO, FLAP, LTA4H, LTA4S, or LTC4S. In some embodiments, the first active agent also decreases any undesired inflammation (e.g., tuberculosis) resulting from administration of the leukotriene inhibitor.

In some embodiments, the first active agent and an IL-1 receptor antagonist treat an inflammatory disorder by (1) decreasing the chemotaxis of leukocytes, and (2) blocking the stimulation of T cell IL-1 receptor. In some embodiments, the first active agent also decreases any undesired inflammation (e.g., pneumonia, and bone and joint infections) resulting from administration of the IL-1 receptor antagonist.

In some embodiments, the first active agent and an IL-2 receptor antagonist treat an inflammatory disorder by (1) decreasing the chemotaxis of leukocytes, and (2) blocking the stimulation of T cell IL-2 receptor. In some embodiments, the first active agent also decreases any undesired inflammation (e.g., gastrointestinal disorders) resulting from administration of the IL-2 receptor antagonist.

In some embodiments, the first active agent and a cytotoxic agent treat an inflammatory disorder by (1) decreasing the chemotaxis of leukocytes, and (2) treating neoplastic disease. In some embodiments, the first active agent also decreases any undesired inflammation (e.g., neutropenia) resulting from administration of the cytotoxic agent.

In some embodiments, the first active agent and an immunomodulatory agent treat an inflammatory disorder by (1) decreasing the chemotaxis of leukocytes, and (2) enhancing, or suppressing the immune system. In some embodiments, the first active agent also decreases any undesired inflammation (e.g., hematologic side effects) resulting from administration of the immunomodulatory agent.

In some embodiments, the first active agent (and an antibiotic treat an inflammatory disorder by (1) decreasing the chemotaxis of leukocytes, and (2) by blocking cell and/or microbial growth by disrupting the cell cycle, or by blocking histone deacetylase. In some embodiments, the first active agent also decreases any undesired inflammation (e.g., cardiotoxicity) resulting from administration of the antibiotic.

In some embodiments, the first active agent and a T-cell co-stimulatory blocker treat an inflammatory disorder by (1) decreasing the chemotaxis of leukocytes, and (2) modulating a co-stimulatory signal which is required for full T-cell activation. In some embodiments, the first active agent also decreases any undesired inflammation (e.g., neutropenia) resulting from administration of the T-cell co-stimulatory blocker.

In some embodiments, the first active agent and a B cell depleting agent treat an inflammatory disorder by (1) decreasing the chemotaxis of leukocytes, and (2) inhibits B-cell activity. In some embodiments, the first active agent also decreases any undesired inflammation (e.g., Progressive Multifocal Leukoencephalopathy) resulting from administration of the B-cell depleting agent.

In some embodiments, the first active agent and an immunosuppressive agent treat an inflammatory disorder by (1) decreasing the chemotaxis of leukocytes, and (2) selectively or non-selectively inhibits or prevents activity of the immune system. In some embodiments, the first active agent also decreases any undesired inflammation (e.g., lymphoma) resulting from administration of immunosuppressive agent.

In some embodiments, the first active agent and an alkylating agent treat an inflammatory disorder by (1) decreasing the chemotaxis of leukocytes, and (2) inducing covalent binding of alkyl groups to cellular molecules. In some embodiments, the first active agent also decreases any undesired inflammation (e.g., immune suppression) resulting from administration of the alkylating agent.

In some embodiments, the first active agent and an anti-metabolite treat an inflammatory disorder by (1) decreasing the chemotaxis of leukocytes, and (2) preventing the biosynthesis or use of normal cellular metabolites. In some embodiments, the first active agent also decreases any undesired inflammation (e.g., mutagenesis) resulting from administration of the anti metabolite.

In some embodiments, the first active agent and a plant alkaloid treat an inflammatory disorder by (1) decreasing the chemotaxis of leukocytes, and (2) interfering with normal microtubule breakdown during cell division. In some embodiments, the first active agent also decreases any undesired inflammation (e.g., leukopenia) resulting from administration of the plant alkaloid.

In some embodiments, the first active agent and a terpenoid treat an inflammatory disorder by (1) decreasing the chemotaxis of leukocytes, and (2) treating neoplastic disease or microbial infections. In some embodiments, the first active agent also decreases any undesired inflammation resulting from administration of the terpenoid agent.

In some embodiments, the first active agent and a topoisomerase inhibitor treat an inflammatory disorder by (1) decreasing the chemotaxis of leukocytes, and (2) modulating the action of cellular topoisomerase enzymes. In some embodiments, the first active agent also decreases any undesired inflammation (e.g., gastrointestinal effects) resulting from administration of the topoisomerase inhibitor.

In some embodiments, the first active agent and an antibody treat an inflammatory disorder by (1) decreasing the chemotaxis of leukocytes, and (2) neutralizing inflammatory cytokines such as, for example, TNF alpha. In some embodiments, the first active agent also decreases any undesired inflammation (e.g., tuberculosis) resulting from administration of the antibody.

In some embodiments, the first active agent and a hormonal therapy treat an inflammatory disorder by (1) decreasing the chemotaxis of leukocytes, and (2) suppressing cytokine release. In some embodiments, the first active agent also decreases any undesired inflammation (e.g., cancer) resulting from administration of the hormone.

In some embodiments, the first active agent and an anti-diabetes therapy treat an inflammatory disorder by (1) decreasing the chemotaxis of leukocytes, and (2) improving sensitivity to insulin in muscle and adipose tissue. In some embodiments, the first active agent also decreases any undesired inflammation (e.g., liver inflammation, pancreatitis) resulting from administration of the anti-diabetes agent.

### Cardiovascular Disorders

In some embodiments, the second active agent is selected from an agent that treats a cardiovascular disorder (the “cardiovascular disorder agent”). In some embodiments, the first active agent rescues a mammal from inflammation partially or fully caused by the cardiovascular disorder agent.

HDL-raising therapies include, but are not limited to, niacin, fibrates, statins, Apo-A1 mimetic peptides (e.g., DF-4, Novartis), apoA-I transciptional up-regulators (e.g., RVX-208, Resverlogix), ACAT inhibitors (e.g., avasimibe; IC-976, Pfizer, MCC-147, Mitsubishi Pharma), CETP modulators, or combinations thereof.

In some embodiments, the cardiovascular disorder agent raises HDL non-selectively. In some embodiments, the cardiovascular disorder agent down-regulates transcription of a CETP gene. In some embodiments, the second active agent is niacin.

In some embodiments, the cardiovascular disorder agent is a statin. In some embodiments, the cardiovascular disorder agent is atorvastatin; cerivastatin; fluvastatin; lovastatin; mevastatin; pitavastatin; pravastatin; rosuvastatin; simvastatin; simvastatin and ezetimibe; lovastatin and niacin, extended-release; atorvastatin and amlodipine besylate; simvastatin and niacin, extended-release; or combinations thereof. In some embodiments, the first active agent and the statin synergistically treat a CVD by (1) decreasing the chemotaxis of leukocytes, (2) decreasing the synthesis of cholesterol, and (3) decreasing any undesired inflammation resulting from administration of the statin. In certain instances, statins induce inflammation. In certain instances, administration of a statin results (partially or fully) in myositis. In certain instances, statin-induced myositis is dose-dependent. In some embodiments, prescribing the first active agent allows (partially or fully) a medical professional to increase the prescribed dosage of statin.

In some embodiments, the cardiovascular disorder agent reduces the risk of developing a cardiovascular disorder in individuals with low HDL with metabolic syndrome. In some embodiments, the cardiovascular disorder agent is a fibrate. In some embodiments, the cardiovascular disorder agent is bezafibrate; ciprofibrate; clofibrate; gemfibrozil; fenofibrate; or combinations thereof. In some embodiments, the first active agent and the fibrate synergistically treat a CVD by (1) decreasing the chemotaxis of leukocytes, and (2) increasing the concentration of HDL. In some embodiments, the first active agent also decreases any undesired inflammation resulting from administration of the fibrate.

In some embodiments, the cardiovascular disorder agent selectively increases the levels of ApoA-I protein (e.g. by transcriptional induction of the gene encoding ApoA-I) and increases the production of nascent HDL (ApoA1-enriched). In some embodiments, the cardiovascular disorder agent is DF4 (Ac-D-W-F-K-A-F-Y-D-K-V-A-E-K-F-K-E-A-F-NH2); DF5; RVX-208 (Resverlogix); or combinations thereof. In some embodiments, the first active agent and the ApoA1 modulator synergistically treat a CVD by (1) decreasing the chemotaxis of leukocytes, and (2) increasing the concentration of HDL. In some embodiments, the first active agent also decreases any undesired inflammation resulting from administration of the ApoA1 modulator.

In some embodiments, the cardiovascular disorder agent is an ACAT inhibitor. In some embodiments, the cardiovascular disorder agent is avasimibe; pactimibe sulfate (CS-505); CI-1011 (2,6-diisopropylphenyl [(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; or combinations thereof. In some embodiments, the first active agent and the ACAT modulator synergistically treat a CVD by (1) decreasing the chemotaxis of leukocytes, and (2) decreasing (a) the production and release of apoB-containing lipoproteins and (b) foam cell formation. In some embodiments, the first active agent also decreases any undesired inflammation resulting from administration of the ACAT inhibitor.

In some embodiments, the cardiovascular disorder agent (partially or completely) the inhibits activity of Cholesteryl Ester Transfer Protein (CETP). In some embodiments, the cardiovascular disorder agent is torcetrapib; anacetrapid; JTT-705 (Japan Tobacco/Roche); or combinations thereof. In some embodiments, the first active agent and the CETP modulator synergistically treat a CVD by (1) decreasing the chemotaxis of leukocytes, and (2) decreasing the transfer cholesterol from HDL cholesterol to LDL. In some embodiments, the first active agent also decreases any undesired inflammation resulting from administration of the CETP inhibitor.

Therapeutics used to treat acute coronary syndrome (ACS) and acute myocardial infarction (AMI) include, but are not limited to, Glycoprotein (GP) IIb/IIIa receptor antagonists, P2Y12 receptor antagonists, and Lp-PLA2-inhibitors.

In some embodiments, the cardiovascular disorder agent is a Glycoprotein (GP) IIb/IIIa receptor antagonist. In some embodiments, the cardiovascular disorder agent is abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)-[1-[3-(piperidin-4-yl)propionyl]piperidin-3-ylcarbonyl]amino]propionic acid trihydrate); or combinations thereof. In some embodiments, the first active agent and the GP IIb/IIIa receptor antagonist synergistically treat a CVD by (1) decreasing the chemotaxis of leukocytes, and (2) inhibiting platelet aggregation. In some embodiments, the first active agent also decreases any undesired inflammation resulting from administration of the GP IIb/IIIa receptor antagonist.

In some embodiments, the cardiovascular disorder agent is a P2Y12 receptor antagonist. In some embodiments, the cardiovascular disorder agent is clopidogrel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences) or combinations thereof. In some embodiments, the first active agent and the P2Y12 receptor antagonist synergistically treat a CVD by (1) decreasing the chemotaxis of leukocytes, and (2) inhibiting platelet aggregation. In some embodiments, the first active agent also decreases any undesired inflammation resulting from administration of the P2Y12 receptor antagonist.

In some embodiments, the cardiovascular disorder agent is an Lp-PLA2 antagonist. In some embodiments, the second active agent is darapladib (SB 480848); SB-435495 (GlaxoSmithKline); SB-222657 (GlaxoSmithKline); SB-253514 (GlaxoSmithKline); or combinations thereof. In some embodiments, the first active agent and Lp-PLA2 antagonist synergistically treat a CVD by (1) decreasing the chemotaxis of leukocytes, and (2) inhibiting the formation of biologically active products from oxidized LDL. In some embodiments, the first active agent also decreases any undesired inflammation resulting from administration of the Lp-PLA2 antagonist.

In some embodiments, the cardiovascular disorder agent is a leukotriene (e.g., LTA4, LTB4, LTC4, LTD4, LTE4, and LTF4) inhibitor (e.g., an antagonist of 5-LO, FLAP, LTA4H, LTA4S, LTA4R; LTB4R; LTB4R1, LTB4R2, LTC4S, LTC4R, LTD4R, LTE4R, CYSLTR1, or CYSLTR2). In some embodiments, the second active agent is an antagonist of 5-LO. In some embodiments, the second active agent is an antagonist of FLAP. In some embodiments, the second active agent is A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AM103 (Amira); AM803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2-methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4-yl)phenoxy)methyl)-1-methyl-2(1H)-quinlolinone); or combinations thereof. In some embodiments, the first active agent (i.e., a MIF antagonist and/or a modulator of an interaction between RANTES and Platelet Factor 4) and a leukotriene antagonist synergistically treat a CVD by (1) decreasing the chemotaxis of leukocytes, and (2) inhibiting the adhesion and activation of leukocytes on the endothelium, decreasing the chemotaxis of neutrophils and reducing the formation of reactive oxygen species. In some embodiments, the first active agent also decreases any undesired inflammation resulting from administration of the leukotriene antagonist.

### Gene Therapy

In some embodiments, are methods and pharmaceutical compositions for modulating a disorder of a cardiovascular system, comprising a synergistic combination of (a) a therapeutically-effective amount of a first active agent selected from (1) a modulator of MIF; (2) a modulator of an interaction between RANTES and Platelet Factor 4; or (3) combinations thereof; and (b) gene therapy.

In some embodiments, the gene therapy comprises modulating the concentration of a lipid and/or lipoprotein (e.g., HDL) in the blood of an individual in need thereof. In some embodiments, modulating the concentration of a lipid and/or lipoprotein (e.g., HDL) in the blood comprises transfecting DNA into an individual in need thereof. In some embodiments, the DNA encodes an Apo A1 gene, an LCAT gene, and/or an LDL gene. In some embodiments, the DNA is transfected into a liver cell.

In some embodiments, the DNA is transfected into a liver cell via use of ultrasound. For disclosures of techniques related to transfecting ApoA1 DNA via use of ultrasound see U.S. Pat. No. 7,211,248, which is hereby incorporated by reference for those disclosures.

In some embodiments, an individual is administered a vector engineered to carry the human gene (the “gene vector”). For disclosures of techniques for creating an LDL gene vector see U.S. Pat. No. 6,784,162, which is hereby incorporated by reference for those disclosures. In some embodiments, the gene vector is a retrovirus. In some embodiments, the gene vector is not a retrovirus (e.g. it is an adenovirus; a lentivirus; or a polymeric delivery system such as METAFECTENE, SUPERFECT®, EFFECTENE®, or MIRUS TRANSIT). In certain instances, a retrovirus, adenovirus, or lentivirus will have a mutation such that the virus is rendered incompetent.

In some embodiments, the vector is administered in vivo (i.e., the vector is injected directly into the individual, for example into a liver cell), ex vivo (i.e., cells from the individual are grown in vitro and transduced with the gene vector, embedded in a carrier, and then implanted in the individual), or a combination thereof.

In certain instances, after administration of the gene vector, the gene vector infects the cells at the site of administration (e.g. the liver). In certain instances the gene sequence is incorporated into the subject's genome (e.g. when the gene vector is a retrovirus). In certain instances the therapy will need to be periodically re-administered (e.g. when the gene vector is not a retrovirus). In some embodiments, the therapy is re-administered annually. In some embodiments, the therapy is re-administered semi-annually. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

### RNAi Therapies

In some embodiments, are methods and pharmaceutical compositions for modulating a disorder of a cardiovascular system, comprising a synergistic combination of (a) a therapeutically-effective amount of a first active agent selected from (1) a modulator of MIF; (2) a modulator of an interaction between RANTES and Platelet Factor 4; or (3) combinations thereof; and (b) silencing the expression of a gene that increases the concentration of a lipid in blood (the “target gene”). In some embodiments, the target gene is Apolipoprotein B (Apo B), Heat Shock Protein 110 (Hsp 110), and Proprotein Convertase Subtilisin Kexin 9 (Pcsk9) (ALN-PCS, BMS-PCSK9_{RX}). In some embodiments, the target gene is C-reactive protein (CRP) (ISIS-CRP_{RX}).

In some embodiments, the target gene is silenced by RNA interference (RNAi). In some embodiments, the RNAi therapy comprises use of an siRNA molecule. In some embodiments, a double stranded RNA (dsRNA) molecule with sequences complementary to an mRNA sequence of a gene to be silenced (e.g., Apo B, Hsp 110 and Pcsk9) is generated (e.g by PCR). In some embodiments, a 20-25 bp siRNA molecule with sequences complementary to an mRNA sequence of a gene to be silenced is generated. In some embodiments, the 20-25 bp siRNA molecule has 2-5 bp overhangs on the 3′ end of each strand, and a 5′ phosphate terminus and a 3′ hydroxyl terminus. In some embodiments, the 20-25 bp siRNA molecule has blunt ends. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, an siRNA molecule is “fully complementary” (i.e., 100% complementary) to the target gene. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target gene. In some embodiments, there is a 1 bp mismatch, a 2 bp mismatch, a 3 bp mismatch, a 4 bp mismatch, or a 5 bp mismatch.

In certain instances, after administration of the dsRNA or siRNA molecule, cells at the site of administration (e.g. the cells of the liver and/or small intestine) are transformed with the dsRNA or siRNA molecule. In certain instances following transformation, the dsRNA molecule is cleaved into multiple fragments of about 20-25 bp to yield siRNA molecules. In certain instances, the fragments have about 2 bp overhangs on the 3′ end of each strand.

In certain instances, an siRNA molecule is divided into two strands (the guide strand and the anti-guide strand) by an RNA-induced Silencing Complex (RISC). In certain instances, the guide strand is incorporated into the catalytic component of the RISC (i.e. argonaute). In certain instances, the guide strand binds to a complementary RB 1 mRNA sequence. In certain instances, the RISC cleaves an mRNA sequence of a gene to be silenced. In certain instances, the expression of the gene to be silenced is down-regulated.

In some embodiments, a sequence complementary to an mRNA sequence of a target gene is incorporated into a vector. In some embodiments, the sequence is placed between two promoters. In some embodiments, the promoters are orientated in opposite directions. In some embodiments, the vector is contacted with a cell. In certain instances, a cell is transformed with the vector. In certain instances following transformation, sense and anti-sense strands of the sequence are generated. In certain instances, the sense and anti-sense strands hybridize to form a dsRNA molecule which is cleaved into siRNA molecules. In certain instances, the strands hybridize to form an siRNA molecule. In some embodiments, the vector is a plasmid (e.g pSUPER; pSUPER.neo; pSUPER.neo+gfp).

In some embodiments, an siRNA molecule is administered in vivo (i.e., the vector is injected directly into the individual, for example into a liver cell or a cell of the small intestine, or into the blood stream).

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering a nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA molecule described herein is administered to the liver by any suitable manner (see e.g., Wen et al., 2004, World J Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66).

In some embodiments, an siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

For disclosures of techniques related to silencing the expression of Apo B and/or Hsp110 see U.S. Pub. No. 2007/0293451 which is hereby incorporated by reference for such disclosures. For disclosures of techniques related to silencing the expression of Pcsk9 see U.S. Pub. No. 2007/0173473, which is hereby incorporated by reference for such disclosures.

### Antisense Therapies

In some embodiments, are methods and pharmaceutical compositions for modulating a disorder of a cardiovascular system, comprising a synergistic combination of (a) a therapeutically-effective amount of a first active agent selected from (1) a modulator of MIF; (2) a modulator of an interaction between RANTES and Platelet Factor 4; or (3) combinations thereof; and (b) inhibiting the expression of and/or activity of an RNA sequence that increases the concentration of a lipid in blood (the “target sequence”). In some embodiments, inhibiting the expression of and/or activity of a target sequence comprises use of an antisense molecule complementary to the target sequence. In some embodiments, the target sequence is microRNA-122 (miRNA-122 or mRNA-122). In certain instances, inhibiting the expression of and/or activity of miRNA-122 results (partially or fully) in a decrease in the concentration of cholesterol and/or lipids in blood.

In some embodiments, an antisense molecule that is complementary to a target sequence is generated (e.g. by PCR). In some embodiments, the antisense molecule is about 15 to about 30 nucleotides. In some embodiments, the antisense molecule is about 17 to about 28 nucleotides. In some embodiments, the antisense molecule is about 19 to about 26 nucleotides. In some embodiments, the antisense molecule is about 21 to about 24 nucleotides. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, the antisense molecules are single-stranded, double-stranded, circular or hairpin. In some embodiments, the antisense molecules contain structural elements (e.g., internal or terminal bulges, or loops).

In some embodiments, an antisense molecule is “fully complementary” (i.e., 100% complementary) to the target sequence. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target RNA sequence. In some embodiments, there is a 1 bp mismatch, a 2 bp mismatch, a 3 bp mismatch, a 4 bp mismatch, or a 5 bp mismatch.

In some embodiments, the antisense molecule hybridizes to the target sequence. As used herein, “hybridize” means the pairing of nucleotides of an antisense molecule with corresponding nucleotides of the target sequence. In certain instances, hybridization involves the formation of one or more hydrogen bonds (e.g., Watson-Crick, Hoogsteen or reversed Hoogsteen hydrogen bonding) between the pairing nucleotides.

In certain instances, hybridizing results (partially or fully) in the degradation, cleavage, and/or sequestration of the RNA sequence.

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering a nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA molecule described herein is administered to the liver by any suitable manner (see e.g., Wen et al., 2004, World J. Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66).

In some embodiments, an siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

For disclosures of techniques related to silencing the expression of miRNA-122 see WO 07/027,775A2, which is hereby incorporated by reference for such disclosures.

### Device-Mediated Therapies

In some embodiments, the device mediated strategy comprises removing a lipid from an HDL molecule in an individual in need thereof (delipidation), removing an LDL molecule from the blood or plasma of an individual in need thereof (delipidation), or a combination thereof. For disclosures of techniques for removing a lipid from an HDL molecule and removing an LDL molecule from the blood or plasma of an individual in need thereof see U.S. Pub. No. 2008/0230465, which is hereby incorporated by reference for those disclosures.

In certain instances, the delipidation therapy will need to be periodically re-administered. In some embodiments, the delipidation therapy is re-administered annually. In some embodiments, the delipidation therapy is re-administered semi-annually. In some embodiments, the delipidation therapy is re-administered monthly. In some embodiments, the delipidation therapy is re-administered semi-weekly. In some embodiments, the therapy is re-administered when the subject's HDL level decreases below about 60 mg/dL. In some embodiments, the therapy is re-administered when the subject's HDL level decreases below about 50 mg/dL. In some embodiments, the therapy is re-administered when the subject's HDL level decreases below about 45 mg/dL. In some embodiments, the therapy is re-administered when the subject's HDL level decreases below about 40 mg/dL. In some embodiments, the therapy is re-administered when the subject's HDL level decreases below about 35 mg/dL. In some embodiments, the therapy is re-administered when the subject's HDL level decreases below about 30 mg/dL.

### Pharmaceutical Compositions

Disclosed herein, in certain embodiments, is a pharmaceutical composition for modulating a disorder of a cardiovascular system, comprising a synergistic combination of (a) a therapeutically-effective amount of a first active agent that inhibits interactions between RANTES and Platelet Factor 4; and (b) a second active agent selected from an agent that treats cardiovascular disorders.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition for modulating a disorder of a cardiovascular system further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions described herein are formulated into any suitable dosage form, including but not limited to, aqueous oral dispersions, liquids, gels, syrups, elixirs, slurries, suspensions and the like, for oral ingestion by a individual to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile release formulations, multiparticulate formulations, and mixed immediate release and controlled release formulations.

### Multi-Particulate Dosage Forms

In some embodiments, the pharmaceutical compositions described herein are formulated as mulitparticulate formulations. In some embodiments, the pharmaceutical compositions described herein comprise a first population of particles and a second population of particles. In some embodiments, the first population comprises an active agent. In some embodiments, the second population comprises an active agent. In some embodiments, the dose of active agent in the first population is equal to the dose of active agent in the second population. In some embodiments, the dose of active agent in the first population is not equal to (e.g., greater than or less than) the dose of active agent in the second population.

In some embodiments, the active agent of the first population is released before the active agent of the second population. In some embodiments, the second population of particles comprises a modified-release (e.g., delayed-release, controlled-release, or extended release) coating. In some embodiments, the second population of particles comprises a modified-release (e.g., delayed-release, controlled-release, or extended release) matrix.

Coating materials for use with the pharmaceutical compositions described herein include, but are not limited to, polymer coating materials (e.g., cellulose acetate phthalate, cellulose acetate trimaletate, hydroxy propyl methylcellulose phthalate, polyvinyl acetate phthalate); ammonio methacrylate copolymers (e.g., Eudragit® RS and RL); poly acrylic acid and poly acrylate and methacrylate copolymers (e.g., Eudragite S and L, polyvinyl acetaldiethylamino acetate, hydroxypropyl methylcellulose acetate succinate, shellac); hydrogels and gel-forming materials (e.g., carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydroxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (m. wt. ^{˜}5 k-5,000 k), polyvinylpyrrolidone (m. wt. ^{˜}10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ^{˜}30 k-300 k), polysaccharides such as agar, acacia, karaya, tragacanth, algins and guar, polyacrylamides, Polyox®polyethylene oxides (m. wt. ^{˜}100 k-5,000 k), AquaKeep® acrylate polymers, diesters of polyglucan, crosslinked polyvinyl alcohol and poly N-vinyl-2-pyrrolidone, sodium starch; hydrophilic polymers (e.g., polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, other acrylic acid derivatives, sorbitan esters, natural gums, lecithins, pectin, alginates, ammonia alginate, sodium, calcium, potassium alginates, propylene glycol alginate, agar, arabic gum, karaya gum, locust bean gum, tragacanth gum, carrageens gum, guar gum, xanthan gum, scleroglucan gum); or combinations thereof. In some embodiments, the coating comprises a plasticiser, a lubricant, a solvent, or combinations thereof. Suitable plasticisers include, but are not limited to, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

In some embodiments, the second population of particles comprises a modified release matrix material. Materials for use with the pharmaceutical compositions described herein include, but are not limited to microcrytalline cellulose, sodium carboxymethylcellulose, hydroxyalkylcelluloses (e.g., hydroxypropylmethylcellulose and hydroxypropylcellulose), polyethylene oxide, alkylcelluloses (e.g., methylcellulose and ethylcellulose), polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate, or combinations thereof.

In some embodiments, the first population of particles comprises a cardiovascular disorder agent. In some embodiments, the second population of particles comprises a (1) a modulator of MIF; (2) a modulator of an interaction between RANTES and Platelet Factor 4; or (3) combinations thereof. In some embodiments, the first population of particles comprises a (1) a modulator of MIF; (2) a modulator of an interaction between RANTES and Platelet Factor 4; or (3) combinations thereof. In some embodiments, the second population of particles comprises a cardiovascular disorder agent.

### Additional Dosage Forms

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations disclosed herein are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

Buccal formulations include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a pharmaceutical compositions disclosed here are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) an active agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an active agent optionally with carriers, optionally a rate controlling barrier to deliver a an active agent to the skin of the host at a controlled and predetermined rate over a prolonged period of time, and means to secure the device to the skin.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, an active agent disclosed herein is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

An active agent disclosed herein is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

### Dosages and Administration

In some embodiments, the pharmaceutical compositions disclosed herein are administered to an individual in need thereof. In some embodiments, the pharmaceutical compositions disclosed herein are administered to an individual diagnosed with (i.e., satisfies the diagnostic criteria for) a cardiovascular disease (e.g., atherosclerosis, angina, stenosis, restenosis, high blood pressure, an aneurysm, an embolism, a blood clot, and/or an infarction (e.g., a myocardial infarction or stroke). In some embodiments, the pharmaceutical compositions disclosed herein are administered to an individual suspected of having a cardiovascular disease. In some embodiments, the pharmaceutical compositions disclosed herein are administered to an individual predisposed to develop a cardiovascular disease.

In certain instances, an individual is at risk of atherosclerosis if their c-reactive protein (CRP) levels are above about 3.0 mg/L. In certain instances, an individual is at risk of atherosclerosis if their homocysteine levels exceed about 15.9 mmol/L. In certain instances, an individual is at risk of atherosclerosis if their LDL levels exceed about 160 mg/dL. In certain instances, an individual is at risk of atherosclerosis if their HDL levels are below about 40 mg/dL. In certain instances, an individual is at risk of atherosclerosis if their serum creatinine levels exceed about 1.5 mg/dL. In certain instances, an individual is pre-disposed to develop atherosclerosis if they possess the “G” allele of SNP rs10757278 and/or the “C” allele of SNP rs1333049 both of which are located at the locus 9p21. For disclosures regarding the “G” allele of SNP rs10757278 and/or the “C” allele of SNP rs1333049 see Science, Jun. 8, 2007; 316(5830):1491-93 which is herein incorporated by reference for such disclosures. In certain instances, an individual is pre-disposed to develop atherosclerosis if they possess LTA4H haplotypes Hap A, HapB, HapC, HapL, HapK, and/or HapQ. For disclosures regarding LTA4H haplotypes see International Publication No. WO/2006/105439 which is herein incorporated by reference for such disclosures.

The daily dosages appropriate for an active agent disclosed herein are from about 0.01 to 3 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the active agents used, the diseases or conditions to be treated, the mode of administration, the requirements of the individual subject, the severity of the disease or condition being treated, and the judgment of the practitioner.

In some embodiments, administration of the cardiovascular disorder agent results in (either partially or fully) undesired inflammation. In some embodiments, the anti-inflammatory agent is administered to the individual to treat the undesired inflammation. In some embodiments, the administration of the cardiovascular agent is discontinued until the inflamed cells and/or tissue is no longer inflamed. In some embodiments, after the inflamed cells and/or tissue are no longer inflamed, administration of the cardiovascular disorder agent recommences. In some embodiments, administration of the cardiovascular agent recommences in combination with an alternative dose of the anti-inflammatory agent.

In the case wherein the individual's condition does not improve, upon the doctor's discretion the administration of an active agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an active agent disclosed herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. An active agent disclosed herein exhibiting high therapeutic indices is preferred. The data obtained from cell culture assays and animal studies are optionally used in formulating a range of dosage for use in human. The dosage of such an active agent disclosed herein lies preferably within a range of circulating concentrations that include the ED50 with minimal toxicity. The dosage optionally varies within this range depending upon the dosage form employed and the route of administration utilized.

### EXAMPLES

### Material and Methods

### Cell Culture

Endothelial cells from the human umbilical cord (HUVEC, human umbilical vein endothelial cells, PromoCell, Heidelberg) are cultivated in Endothelial Cell Growth Medium (PromoCell, Heidelberg) and used after 2 to 4 passages.

Monocyte Mono Mac 6-cells (MM6, DSMZ) are cultivated in RPMI 1640 Medium (PAA Laboratories, Pasching, Austria) with addition of 10% fetal calf serum, 2 mM of L-glutamine (Biowhittaker), 1 mM of sodium pyruvate, 50 μg/ml of Gentamycin and 9 μg/ml of insulin (MM6 medium). The cells are seeded with a density of 2×10⁵/ml in 2 ml of MM6 medium in 24 well plates and are cultivated at 37 degrees C. in a humidified atmosphere with 5% CO₂ for 3 to 4 days, before they are used for experiments.

### Peptides

Peptides of the sequence SEQ ID NO: 3 per formula (3), its mouse orthologue, as well a control peptide of sequence are chemically synthesized by means of t-Boc based solid phase peptide synthesis making use of 4-methyl benzhydrylamine resin, purified by means of reverse-phase HPLC, and optionally formed into a ring in 6 M of guanidine HCl/Tris pH 8. The molecular mass is determined by means of electrospray mass spectrometry (Dawson P E, Kent S B. (2000) Annu Rev Biochem. 69: 923-960, Hackeng T M, Griffin J H, Dawson P E. (1999) Proc Natl Acad Sci U.S.A., Vol 96, p. 10068-10073).

### Example 1

Plasmon resonance studies are used to analyze the inhibitory effect of the peptide of sequence SEQ ID NO: 3 per formula (3) on the formation of heteroaggregates of RANTES and PF4. The plasmon resonance studies is carried out using HBS-EP buffer (10 mM HEPES, 150 mM NaCl, 0.005% Tween 20, pH 7.4). Two flow cells of a C1 chip (Biacore AB, Uppsala, Sweden) are activated by injection of 50 μl of ethyl(dimethylaminopropyl)carbodiimide/N-hydroxy-succinimide (0.2 M/0.05 M, Pierce Co.) and then 20 μl of Streptavidine (0.2 mg/ml, Sigma-Aldrich) os perfused over the activated surface. After this, the surface is inactivated by four consecutive injections of 20 μl ethylene diamine (1 M, pH 8, Sigma-Aldrich).

At the N-terminus, biotinylated human PF4 (bPF4) is chemically synthesized by means of t-Boc based solid phase peptide synthesis and native chemical ligation of PF4 (Dawson P E, Kent S B. (2000) Annu Rev Biochem. 69: 923-960, Hackeng T M, Griffin J H, Dawson P E. (1999) Proc Natl Acad Sci USA, Vol. 96, p. 10068-10073). The bPF4 is immobilized on the dextran surface of a C1 sensor chip by injecting 200 μg/ml of bPF4 in HBS-EP across one of the flow chambers and registering 240 resonance units (RU). The second flow chamber is not treated with bPF4 and serves as a reference.

The binding to bPF4 to RANTES (0.5 μM, recombinant human RANTES, Peprotech, Rocky Hill, N.J., USA) or RANTES (0.5 μM) that is preincubated with various concentrations, 0 μM, 10 μM, 50 μM and 100 μM, of the peptide of sequence SEQ ID NO: 3 per formula (3) in HBS-EP buffer over night at room temperature is determined by means of injection of 15 μl of the particular peptide/RANTES mixture and observation of the binding for 180 seconds. The coupling sequence and the measurements are carried out in a Biacore 2000 (Biacore AB) device at a flow rate of 5 μl/min. Sensorgrams of the RANTES binding are corrected for nonspecific background signals by means of the software BIAevaluation 3.0 (Biacore AB) and equilibrium resonance units (RU) is determined for each injection.

### Example 2

### Inhibition of the Monocyte Arrest on Activated Endothelium

The interaction of monocyte Mono Mac 6 cells on activated endothelial cells is investigated as follows: Petri dishes with confluent HUVEC cell layers, which are activated with IL-1B, Peprotech, 10 ng/ml, 12 hours), are placed in a flow chamber. Mono Mac 6 cells (0.5×10⁶ cells per ml) are resuspended in properly proportioned Hank solution (HBSS with 10 mM Hepes (Gibco BRL), pH 7.3, 0.5% bovine serum albumen (Serva) and kept on ice. Five minutes before the experiment, there is added to the monocyte MM6 cells Ca²⁺ and Mg²⁺ to a final concentration of 1 mM each and 60 nM of the chemokines RANTES (Peprotech, Rocky Hill, N.J., USA) and PF4 (ChromaTec, Greifswald) and 6 μM each of the peptides of SEQ ID NO: 2 per formula (2), sequence SEQ ID NO: 3 per formula (3), or a control peptide and the materials are heated to 37 degrees C. The thus pretreated cells are then perfused across the endothelial cells at 1.5 dyn/cm3 on a microscope of type IX 50 of the Olympus Co. The number of monocytes that are adherent by interaction with the endothelial cells is determined after 4 minutes in various fields by means of image analysis of pictures of a video camera (3CCD, JVC) and recorder. The data are evaluated as mean (n=5)±standard deviation (p<0.02) against a control.

### Example 3

### In Vivo Investigations in a Mouse Model of Atherosclerosis

Female ApoE−/− littermate mice 9 to 12 weeks old (The Jackson Lab, Bar Harbor, Me., USA) will serve as the model for atherosclerosis. These are given a fat-rich diet (21% fat; Altromin C1061) for 12 weeks. During this time, two groups of mice receive thrice weekly intraperitoneal injections of 50 μg of peptide of sequence SEQ ID NO: 8 per formula (9), given below:

CKEYFYTSSKSSNLAVVFVTRC (8)(SEQ ID NO: 8)

(n=12 mice) or of a control peptide of sequence SEQ ID NO: 9 per formula (9), as given below:

KEYFYTSGK (9)(SEQ ID NO: 9)

(n=7 mice) in saline solution. An untreated group of mice (n=12) serve as an additional control.

The mice are sacrificed for histological studies. During the period of the experiment, the mice are maintained healthy. Blood samples are taken at the start and after the end of the experimental feeding. The leukocyte count is determined by hemocytometry and the sera are collected and the cholesterol level is determined by means of Infinity Cholesterol kits (Thermo Electron, Melbourne, Australia).

The extent of the atherosclerosis is determined at the aortal roots and thoracoabdominal aortas by staining the lipid deposits with oil red O stain (Veillard N R, Kwak B, Pelli G, Mulhaupt F, James R W, Proudfoot A E, Mach F. Antagonism of RANTES receptors reduces atherosclerotic plaque formation in mice. Circ Res. 2004; 94: 253-61) and is quantified by means of computerized image analysis (Diskus software, Hilgers, Aachen). Regions of atherosclerotic lesions are determined in 5 micron transverse sections through heart and aortal root. The determination is done for each aortal root by means of lipid-stained regions of 6 sections, at a distance of 50 μm from each other. The regions of atherosclerotic lesions re divided by the entire surface of the valve of each section. The thoracoabdominal aorta is opened along the ventral midline and the regions of lesions re stained in an en face preparation by means of oil red O staining. The proportion of lipid deposition is calculated as the stained region divided by the entire thoracoabdominal surface.

### Example 4

### Preparation of Multi-Particulate Dosage Form

A multiparticulate dosage form is prepared. The dosage form comprises an immediate release population of particles containing lovastatin. The dosage form further comprises a controlled-release population of particles comprising the peptide of SEQ ID NO: 2.

10 kg of lovastatin, 23 kg of lactose, 0.7 kg of croscarmellose sodium, 0.7 kg polyvinylpyrrolidone K25 are blended in a high-speed blender. The dry mixture is granulated with 4.3 kg of granulating solution (dissolve 0.02 kg of BHA in 1.7 kg of ethanol while mixing in the high-speed blender and add 2.6 kg of demineralized water to the resulting solution). The granulation is dried in a bed-fluid dryer. The dried granulation is sieved in a 0.5 mm sieve to obtain granulation particles of the desired size.

5 mg of COR100140 26 kg of lactose, 0.8 kg of croscarmellose sodium, 0.8 kg polyvinylpyrrolidone K25 are blended in a high-speed blender. The dry mixture is granulated with 34.3 kg of granulating solution (dissolve 0.02 kg of BHA in 1.7 kg of ethanol while mixing in the high-speed blender and add 2.6 kg of demineralized water to the resulting solution). The granulation is dried in a bed-fluid dryer. The dried granulation is sieved in a 0.5 mm sieve to obtain granulation particles of the desired size. The granules are then sprayed with a controlled—release coating composition comprising.

The immediate release granules and the controlled-release granules are mixed together. The resulting mixture is encapsulated in gelatine capsules.

### Example 5

### Preparation of a Multi-Particulate Dosage Form

10 kg Methotrexate is first screened through a suitable screen (e.g. 500 micron). 25 kg Lactose monohydrate, 8 kg hydroxypropylmethyl cellulose, the screened methotrexate and 5 kg calcium hydrogen phosphate (anhydrous) are then added to a suitable blender (e.g. a tumble mixer) and blended. The blend is screened through a suitable screen (e.g. 500 micron) and reblended. About 50% of the lubricant (2.5 kg, magnesium stearate) is screened, added to the blend and blended briefly. The blend is roller compacted through a suitable roller compactor. The ribbon blend is then granulated, by screening through a suitable screen (e.g. 500 micron) and reblended. The remaining lubricant (2 kg, magnesium stearate) is screened, added to the blend and blended briefly. The granules are screened (e.g. 200 micron) to obtain granulation particles of the desired size.

Peptide granules are prepared by blending 2.8 kg of the peptide of SEQ ID NO: 2 with microcrystalline cellulose (Avicel® PH101, FMC Corp., Philadelphia, Pa.) in relative amounts of 95:5 (w/w), wet massing the blend in a Hobart mixer with water equivalent to approximately 27% of the weight of the blend, extruding the wet mass through a perforated plate (Luwa EXKS-1 extruder, Fuji Paudal Co., Osaka Japan), spheronizing the extrudate (Luwa QJ-230 marumerizer, Fuji Paudal Co.) and drying the final granules which are about 1 mm diameter. The granules are optionally coated with a plasticized ethylcellulose dispersion (Surelease®, Colorcon, West Point, Pa., typically applied at 15% solids concentration) in a bottom spray Wurster fluid bed coater (Aeromatic Strea-1, Niro Inc., Bubendorf, Switzerland) to obtain sustained release granules. The amount of coating applied is varied to obtain different dissolution rate behavior. For example, an additional coating of 2% Opadry® is optionally applied over the Surelease® Coat.

The methotrexate immediate release granules and the peptide of SEQ ID NO: 2 sustained release granules are mixed together and the resulting mixture is encapsulated in gelatin capsules.

### Example 6

### Toxicity Study Following Statin/the Peptide of SEQ ID NO: 2 Combination in Mouse Model

### Study Design

Female Harlan Sprague-Dawley mice weighing 20 to 24 g are used. The animals used were within an age range of 6 to 8 weeks at the start of dosing.

The mice are divided into two groups: the experimental group (n=16) and the control group (n=16). The experimental group receives daily intraperitoneal injections of a combination of simvastatin (80 mg/kg) and the peptide of SEQ ID NO: 2 (1.5 mg/kg) (n=16 mice) for 14 days. The experimental group receives daily intraperitoneal injections of a saline solution (n=16 mice) for 14 days.

The mice are sacrificed for histological studies. Four mice from the experimental group are sacrificed on each of days 5, 7, 12, and 14. Four mice from the control group are sacrificed on each of days 5, 7, 12, and 14.

### Necropsy and Histology

Tissue sample are taken from the (a) heart, (b) kidneys, (c) liver, (d) stomach, and (e) muscle tissues. The sampled muscles tissues are taken from (a) the right fore limb (the biceps femoris, extensor digitorum longus, tibialis cranialis, and vastus medialis); (b) the left hind limb (the biceps brachii, extensor carpi radialis longus, and flexor carpi ulnaris); the abdominal peritoneal; the diaphragm; the masseter superficialis; the tongue; and the trapezius).

Tissues are fixed in buffered 10% formalin, processed to wax blocks, and then sectioned and stained with haematoxylin and eosin for examination by light microscopy. Necrosis is graded subjectively. Minimal necrosis is up to 10 necrotic fibers in the whole section; mild is up to about 20% necrotic fibers; moderate is up to about 50% necrotic fibers; and severe is more than 50% necrotic fibers.

### Electron Microscopy

Samples for ultrastructural assessment are immersion fixed in 2.5% glutaraldehyde fixative. Glutaraldehyde-fixed samples are postfixed in 1% osmium tetroxide and processed to Araldite resin blocks. Thin, 70-90-nm resin sections are cut and stained using uranyl acetate and lead citrate. Ultrastructural morphology is examined with a TEM.

### Muscle Histochemistry

Muscle samples are trimmed, orientated on a cork disk, and frozen in isopentane (Fisher Scientific) pre-cooled with liquid nitrogen. Serial cryosections of 7-μm thickness are cut from each sample for fiber typing. Sections are stained for mATPase activity following pre-incubation at high and low pH. One section is placed in an incubating solution at pH 9.4 consisting of 0.5% ATP (Sigma) in 0.1 M glycine/NaCl buffer with 0.75 M CaCl₂ for 45 minutes at 37° C. A further section is pre-incubation in 0.1 M sodium acetate buffer with 10 mM ETDA (pH 4.1-4.3) for 10 minutes at 4° C. before placing in the incubation solution noted previously. Following incubation the slides are transferred to 2% CoCl₂ for 5 minutes followed by 30 seconds in 10% ammonium sulphide solution. Sections are washed thoroughly in distilled water between each step. Sections are lightly counterstained with Carazzi's haematoxylin before being dehydrated, cleared, and mounted in Histomount.

### Muscle Immunohistochemistry

Serial cryostat sections are stained for fast and slow myosin heavy chains using antibodies (e.g., NCL-MHCf for fast myosin heavy chains, and NCL-MHCs for slow myosin heavy chains). The sections are incubated in the primary antibody for 60 minutes, then incubated in the secondary antibody (i.e., rabbit anti-mouse HRP conjugate) for 30 minutes, before being visualized by incubation with 3,3 diaminobenzidine tetrahydrochloride for 5 minutes. All incubations are at room temperature, and sections are washed thoroughly in tris-buffered saline between each step. Sections are counterstained with Carazzi's haematoxylin before being dehydrated, cleared, and mounted in Histomount. Dewaxed sections are subjected to 2 minutes' full pressure in a microwave pressure cooker containing 0.01 M citrate buffer at pH 6.0, and then 5 minutes' digestion at room temperature by proteinase K. Endogenous peroxidase activity is blocked by incubation in a peroxidase inhibitor for 20 minutes, followed by 15 minutes in 20% normal rabbit serum. Mouse monoclonal antibody is applied for 30 minutes, followed by 30 minutes in peroxidase-conjugated rabbit anti-mouse antibody. Vector Laboratory's SG peroxidase substrate kit (SK4700) is then applied for 10 minutes. Following an additional 15 minutes of incubation in 20% normal rabbit serum, a mouse mAB to fast myosin is applied. This is visualized using Vector Red alkaline phosphatase substrate kit (Vector Labs SK5100) for 10 minutes. All incubations were at room temperature, and sections are washed thoroughly in tris-buffered saline between each step. Sections are dehydrated, cleared, and mounted in Histomount.

### Example 7

### Statin/the Peptide of SEQ ID NO: 2 Combination in Mouse Model of Atherosclerosis

Female ApoE−/− littermate mice 9 to 12 weeks old (The Jackson Lab, Bar Harbor, Me., USA) will serve as the model for atherosclerosis. These are given a fat-rich diet (21% fat; Altromin C1061) for 12 weeks. During this time, two groups of mice receive thrice weekly intraperitoneal injections of a combination of simvastatin (5 mL/kg) and the peptide of SEQ ID NO: 2 (1.5 mg/kg) (n=12 mice) or a saline solution (n=7 mice).

The mice are sacrificed for histological studies. During the period of the experiment, the mice are maintained healthy. Blood samples are taken at the start and after the end of the experimental feeding. The leukocyte count is determined by hemocytometry and the sera are collected and the cholesterol level is determined by means of Infinity Cholesterol kits (Thermo Electron, Melbourne, Australia).

The extent of the atherosclerosis is determined at the aortal roots and thoracoabdominal aortas by staining the lipid deposits with oil red O stain (Veillard N R, Kwak B, Pelli G, Mulhaupt F, James R W, Proudfoot A E, Mach F. Antagonism of RANTES receptors reduces atherosclerotic plaque formation in mice. Circ Res. 2004; 94: 253-61) and is quantified by means of computerized image analysis (Diskus software, Hilgers, Aachen). Regions of atherosclerotic lesions are determined in 5 micron transverse sections through heart and aortal root. The determination is done for each aortal root by means of lipid-stained regions of 6 sections, at a distance of 50 μm from each other. The regions of atherosclerotic lesions re divided by the entire surface of the valve of each section. The thoracoabdominal aorta is opened along the ventral midline and the regions of lesions re stained in an en face preparation by means of oil red O staining. The proportion of lipid deposition is calculated as the stained region divided by the entire thoracoabdominal surface.

### Example 8

### Human Clinical Trial of P4/RANTES Antagonist in Combination with Torcetrapib as a Treatment for Hypercholesterolemia

Study Objective(s): The primary objective of this study is to assess the efficacy of a combination of torcetrapib and the peptide of SEQ ID NO: 2 (C-KEYFYTSGKCSNPAVVFVTR-C) (T/P2; 60 mg/1.5 mg/kg) in subjects with homozygous familial hypercholesterolemia (HoFH) versus torcetrapib (60 mg) alone.

### Methods

Study Design: This study is a prospective, double-blind, multicenter, parallel-treatment trial comparing T/P2 versus T alone in male and female subjects≧18 years of age with HoFH. After initial screening, eligible subjects enter a 4-week screening period, consisting of 2 visits (Weeks −4 and −1), during which all lipid-lowering drugs are discontinued (except for bile acid sequestrants and cholesterol absorption inhibitors) and therapeutic lifestyle change counseling (TLC) according to National Cholesterol Education Program (NCEP) Adult Treatment Panel (ATP-III) clinical guidelines or equivalent is initiated. Subjects already on apheresis continue their treatment regimen maintaining consistent conditions and intervals during the study. At Visit 3 (Week 0), subjects begin treatment with the T/P2 fixed combination once daily (QD) for 6 weeks or T alone. Final visit (Visit 6) occurs at Week 18. Study visits are timed with subjects' apheresis treatments to occur immediately before the visit procedures, where applicable. When the intervals between aphereses are misaligned with a study drug treatment period, the subjects are kept in the same drug treatment period until the next scheduled apheresis, and until the intervals are brought back to the original length of time. Efficacy measures are done at least 2 weeks after the previous apheresis and just before the apheresis procedure scheduled for the day of study visit.

Number of Subjects: 50 subjects divided into two groups—the experimental group (n=25) and the control group (n=25).

Diagnosis and Main Criteria for Inclusion: Men and women 18 years of age or older with definite evidence of the familial hypercholesterolemia (FH) homozygote per World Health Organization guidelines, and with serum fasting triglyceride (TG)≦400 mg/dL (4.52 mmol/L) for subjects aged >20 years and 200 mg/dL (2.26 mmol/L) for subjects aged 18-20 years, are screened for study participation.

Study Treatment: Subjects are randomized into two groups. During the three 6-week treatment period, subjects in the experimental group take 1 tablet of T/P2 QD, with food, immediately after the morning meal. Subjects in the control group take 1 tablet of T QD, with food, immediately after the morning meal.

Efficacy Evaluations: The primary endpoints are the mean percent changes in HDL-C and LDL-C from baseline to the end of each treatment period (ie, Weeks 6, 12 and 18). A lipid profile which included HDL-C and LDL-C is obtained at each study visit.

Safety Evaluations: Safety is assessed using routine clinical laboratory evaluations (hematology and urinalysis panels at Weeks −4, 0 and 18, and chemistry also at Weeks 6 and 12). Vital signs are monitored at every visit, and physical examinations and electrocardiograms (ECGs) are performed at Weeks 0 and 18. Urine pregnancy testing is carried out at every visit except Week −1. Subjects are monitored for adverse events (AEs) from Week 0 to Week 18. Week 18 safety assessments are completed at early termination if this took place.

Statistical Methods: The primary efficacy endpoints are the percent changes in HDL-C and LDL-C from baseline to the end of each treatment period (ie, Weeks 6, 12, and 18). The primary efficacy analysis population is the full analysis set (FAS) which includes all subjects who received at least 1 dose of study drug and had both a baseline and at least 1 valid post-baseline measurement at each analysis period.

The primary efficacy endpoints are analyzed through the computation of sample means of percent (or nominal) changes, their 95% confidence intervals (CIs), 1-sample t-test statistics, and corresponding p-values. Incremental treatment differences between different dose levels are also estimated and 95% CIs obtained. Hypothesis testing is 2-sided with an overall family-wise type I error rate of 5% (ie, p=0.05 significance level). Hochberg's procedure is used to control the family-wise error rate for multiple comparisons.

### Example 9

### Human Clinical Trial of MIF Antagonist in Combination with Atorvastatin as a Treatment for Atherosclerosis

Study Objective(s): To measure the effect of 18 months of treatment with lipid lowering treatment (atorvastatin 80-mg daily) versus 8 months of treatment with atorvastatin in combination with a peptide of SEQ ID NO: 2 (1.5 mg/kg) on coronary artery plaque using intravascular ultrasound (IVUS) imaging of the coronary arteries.

### Study Design:

This study is a prospective, double-blind, multicenter, parallel-treatment trial comparing the effects of atorvastatin 80-mg versus atorvastatin in combination with (80-mg daily) a peptide of SEQ ID NO: 2 (1.5 mg/kg) as measured by IVUS.

The study consists of three phases: (1) subject identification and cardiac catheterization, (2) screening phase to determine eligibility, which includes a 2-week Placebo Run-in Period, and (3) an 18-month, randomized, double-blind treatment phase.

The study includes a total of up to 12 visits (nine required plus three optional) at which safety and/or efficacy assessments are performed: Qualifying IVUS Visit (Cath 1), Screening Visit 1 (SV1), Optional Screening Visits (SV2 and SV3), Randomization Visit (RV), and Clinic Visits for Month 3 (M3), M6, M9, M12, M15, M17 (optional), and M18.

The primary efficacy parameter is percent change in total plaque (atheroma) volume (TPV) by IVUS.

Secondary efficacy parameters include nominal change in TPV and change in percent plaque (atheroma) volume (PPV).

### Number of Patients:

Approximately 400 subjects (200 subjects per treatment group) are to be enrolled

### Diagnosis and Main Criteria for Inclusion:

Male and female subjects between 30-75 years of age with CAD who have had a coronary catheterization. Precise angiographic inclusion criteria will determine subject eligibility, specifically the presence of at least one obstruction in a major cardiac vessel with at least a 20% luminal diameter narrowing by visual estimation. In addition, subjects must have had a “target vessel” for IVUS interrogation with no more than 50% luminal narrowing throughout a segment that was a minimum of 30 mm in length (the “target segment”). The target vessel must not have undergone previous intervention, nor have been a candidate for intervention at the time of Baseline catheterization. Lipid entry criterion require subjects to have a low-density lipoprotein cholesterol (LDL-C) between 125 and 210 mg/dL following a 4- to 10-week washout period if the subject is taking antihyperlipidemic medication.

### Study Treatment:

Subjects are divided into the groups. The first group (n=200) receives atorvastatin. The second group (n=200) receives atorvastatin in combination with a peptide of SEQ ID NO: 2 (1.5 mg/kg).

Placebo Run-in Period: Subjects in the two groups are instructed to take two placebo tablets at bedtime each day and return to the Clinic in two weeks for the Randomization Visit. The time between visits during the Placebo Run-in Period is not to exceed 17 days. Subjects are also required to be at least 90% compliant before randomization to the double-blind period.

Double-Blind Period: Subjects in group 1 are instructed to take 80-mg atorvastatin (2×40-mg tablet) and one placebo tablet daily at bedtime each day for 18 months. Subjects in group 2 are instructed to take 80-mg atorvastatin (2×40-mg tablet) in combination with a peptide of SEQ ID NO: 2 (1.5 mg/kg; 1 tablet) daily at bedtime each day for 18 months.

### Efficacy Evaluations:

Primary efficacy variable: The percent change in total plaque volume for all slices of anatomically comparable segments of the target coronary artery from Baseline to Month 18 measured by IVUS.

Safety Evaluations: Safety of the treatment is assessed by an evaluation of type, frequency, intensity, and duration of all reported adverse events (AEs), monitoring of laboratory parameters, and changes in vital signs. Data for electrocardiogram (ECG) results and physical examination findings is collected.

### Example 10

### In Vivo Investigations in a Rat Model of Arthritis Disease to Test Combination of Etanercept and the Peptide of SEQ ID NO: 2

31 Male Lewis rats are immunized with complete Freund's adjuvant on day 0 to induce an aggressive arthritis characterized by joint destruction and paw swelling.

From day 8 to 20, two groups of rats receive thrice weekly intraperitoneal injections of 50 μg of peptide of SEQ ID NO: 3 (n=12 rats). During this time, the rats also receive weekly subcutaneous injections of 50 μg Etanercept. An untreated group of rats (n=12) serve as a control.

Every week, paw swelling is determined by water displacement plethysmometry. The extent of arthritis is determined at the end of the study on day 21. Radiographs are obtained of the right hind paw to assess bone changes using a semi-quantitative scoring system: demineralization (0-2+), calcaneal erosion (0-1+), and heterotropic bone formation (0-1+), with a maximum possible score=6. Blood samples are tested for neutropenia.

### Example 11

### In Vivo Investigations in a Rat Model of Crohn's Disease to Test Combination of Methotrexate and the Peptide of SEQ ID NO: 2

A modified animal model disclosed in Kirkil, C. et al., J Gastrointest Surg. 2008, 12, 1429-35 is used. Twenty-eight Sprague-Dawley rats are divided into four groups. Groups I and II are used as sham-operated and control groups, respectively. Bowel inflammation is induced by intrajejunal injection of iodoacetamide in groups III and IV. Rats in group IV are treated with oral preparation of methotrexate (10 mg) and intravenous injection of 50 μg of peptide of sequence SEQ ID NO: 3 (n=12 rats).

Three days after induction of the inflammation, partial resection of test loop and anastomosis is performed. Re-laparotomy is performed, anastomosis bursting pressures and peritonitis scores are measured, and tissue samples are obtained for the measurements of tissue hydroxylproline level and mucosal damage index 4 days later.

On the fourth day, measurements of tissue hydroxylproline level and mucosal damage index are obtained. The severity of iodoacetamide induced intestinal inflammation, wound healing in the inflamed intestinal tissue, and decrease in severity of peritonitis is also recorded.

### Example 12

### Human Clinical Trial in SLE to Test Combination of Cyclophosphamide and the Peptide of SEQ ID NO: 2

Study Objective(s): The primary objective of this study is to assess efficacy of the fixed combination cyclophosphamide and the peptide of SEQ ID NO: 2 (C/P2; 60/20 mg, 60/40 mg, 60/80 mg) in subjects with systemic lupus erythematosus (SLE) who are currently receiving cyclophosphamide. This study will also determine if P2 is effective in decreasing disease activity in these patients.

### Methods

The first part of the study is a dose-escalation study in which participants will receive one of two doses of P2 (20 mg, or 40 mg,); this part of the study will last 60 days. At screening, patients will have an IV catheter inserted into their arms for administration of cyclophosphamide and P2. Patients will also have medical and medication history assessments, a comprehensive physical exam, and blood and urine tests. There are 5 study visits for the first part of the trial; these will occur at screening, at study entry, and Days 1, 14, and 28. Selected visits will include physical exam, vital signs measurement, blood and urine tests, and disease activity assessment. At Days 7 and 60, patients will be contacted by phone to report their medication history and any adverse effects they have experienced.

The second part of the study will evaluate a single 80 mg dose of P2; this part of the study will last 90 days. In the study, participants will be randomly assigned to one of two groups. At the start of the study, Group 1 participants will receive P2 and cyclophosphamide and Group 2 participants will receive cyclophosphamide only. There will be 9 study visits; these will occur at study screening, study entry, and Days 1, 4, 7, 14, 28, and 60. At selected visits, patients will undergo physical exam, vital signs measurement, blood tests and urine tests, and disease activity assessment.

Number of Subjects: It is planned to recruit between 30 and 50 subjects for each part of the study.

Diagnosis and Main Criteria for Inclusion: Diagnosis of SLE by American College of Rheumatology (ACR) criteria

Concurrent treatment with intravenous cyclophosphamide for at least one of the following manifestations of lupus: World Health Organization (WHO) class III, IV, or V lupus nephritis; British Isles Lupus Assessment Group (BILAG) score of A for vasculitis; BILAG score of A for cytopenia; BILAG score of A for nervous system; Stable medication regimen for at least 4 weeks prior to study entry; Weight between 40 kg (88.2 lbs) and 125 kg (275.6 lb).

Study Treatment: During the study periods, subjects will have an IV catheter inserted into their arms for intravenous bi-weekly administration of cyclophosphamide and P2.

Efficacy Evaluations: The primary endpoint is SLE disease activity as measured by blood tests, urine tests, and disease activity assessment.

Safety Evaluations: Safety is assessed using routine clinical laboratory evaluations (lupus serology and renal function).

### Example 13

### Human Clinical Trial in Rheumatoid Arthritis to Test Combination of Infliximab and the Peptide of SEQ ID NO: 2

Study Objective(s): The primary objective of this study is to assess efficacy of the fixed combination infliximab/The peptide of SEQ ID NO: 2 (I/P2; 5 mg/kg/20 mg, 10 mg/kg/20 mg, 15 mg/kg/20 mg) in subjects with rheumatoid arthritis who are currently receiving infliximab for treatment of rheumatoid arthritis. This study will also determine if P2 is effective in decreasing disease activity in these patients.

### Methods

Participants will receive nine infusions of infliximab and P2 every three weeks during this 28-week study. The drug is given intravenously (IV, into a vein) over 2 hours. The first three infusions will be at a dose of 5 mg/kg of body weight. Patients will also receive 20 mg P2 in a saline solution (IV, into a vein) over 1 hour. Patients who improve on this regimen will receive another 6 infusions at the same dose. Patients who do not significantly improve on 5 mg/kg at the end of 6 weeks (the third infusion) may continue with phase 2 of the study, in which they will be randomly assigned to receive either: 1) 6 additional doses of tinfliximab at 5 mg/kg per dose, or 2) a gradually increased dose of inflilximab to a maximum of 15 mg/kg. In addition, all patients will continue to take P2 at the same dose as when they entered the study.

Patients will have imaging studies x-rays, MRI and Dexa scan) at the beginning and end of the study and will collect a 24-hour urine sample before each infliximab and P2 infusion.

Number of Subjects: It is planned to recruit between 30 and 50 subjects for each part of the study.

Inclusion criteria: Patients must be at least 18 years old at the screening visit. Patients must have a diagnosis of adult-onset RA of at least six months duration but not longer than fifteen years as defined by the 1987 American College of Rheumatology classification criteria.

Patients must have active RA disease as defined by: 9 tender joints at Screening and Baseline, 9 swollen joints at Screening and Baseline. and fulfilling 1 of the following 2 criteria during the screening period, 30 mm/hour ESR (Westergren), or CRP>15 mg/L.

Patients must have received treatment with infliximab for at least 6 months prior to the Baseline visit. The dose of infliximab and route of administration must have been stable for at least 2 months prior to the baseline visit. The minimum stable dose of infliximab allowed is 5 mg/kg weekly.

Exclusion criteria: Patients must not have a diagnosis of any other inflammatory arthritis (e.g., psoriatic arthritis or ankylosing spondylitis), Patients must not have a secondary, non-inflammatory type of arthritis (e.g. OA or fibromyalgia), Female patients who are breast feeding, pregnant, or plan to become pregnant during the trial or for three months following last dose of study drug, Patients with a history of tuberculosis or positive chest X-ray for tuberculosis or positive, Patients at a high risk of infection (e.g. leg ulcers, indwelling urinary catheter and persistent or recurrent chest infections and patients who are permanently bed ridden or wheelchair bound), Patients with known human immunodeficiency virus (HIV) infection, Patients with an active malignancy of any type or a history of malignancy (except basal cell carcinoma of the skin that has been excised prior to study start), Patients with a current or recent history, as determined by the Investigator, of severe, progressive, and/or uncontrolled renal, hepatic, hematological, gastrointestinal, endocrine, pulmonary, cardiac, neurological, or cerebral disease which would interfere with the patient's participation in the trial, Patients with a history of, or suspected, demyelinating disease of the central nervous system (e.g. multiple sclerosis or optic neuritis).

Primary Outcome measures: Compare efficacy of two dose regimens of infliximab in combination with P2 to infliximab alone in patients with RA measured by the ACR20 at week 28.

Secondary outcome measures: Assess Safety and Tolerability of two dose regimens of infliximab in combination with P2 and infliximab alone in patients with RA; prevention of joint damage in patients with RA; Health Outcomes Measures

Study treatment: During the study periods, subjects will have an IV catheter inserted into their arms for intravenous administration of infliximab and P2.

Efficacy evaluations: The primary endpoint is rheumatoid arthritis disease activity as measured by blood tests, urine tests, x-rays and disease activity assessment.

Safety Evaluations: Safety is assessed using routine clinical laboratory evaluations (blood tests, urine tests).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An isolated peptide, its pharmacologically acceptable salts, derivatives, and conjugates,
characterized in that the peptide has an amino acid sequence SEQ ID NO: 1, as indicated below:
(SEQ ID NO: 1)
C-X1-X2-YFYTS-X3-X4-X5-X6-X7-X8-X9-X10-X11-X12-X13-X14-X15-C
where:
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine;
X15 is chosen from the group containing arginine, lysine, alanine, glutamine, histidine and asparagine, or an amino acid deletion.

2. The peptide of claim 1, characterized in that the peptide has an amino acid sequence SEQ ID NO: 2, as indicated below:
C-KEYFYTSGKCSNPAVVFVTR-C.

3. The peptide of claim 1, characterized in that the peptide has an amino acid sequence SEQ ID NO: 3, as indicated below:
C-KEYFYTSSKCSNLAVVFVTR-C.

4. The peptide of claim 1, characterized in that the peptide has an amino acid sequence SEQ ID NO: 4, as indicated below:
C-QEYFYTSSKCSMAAVVFITR-C.

5. The peptide of claim 1, characterized in that the peptide has an amino acid sequence SEQ ID NO: 13, as indicated below:
(SEQ ID NO: 13)
C-KEYFYTSSKSSNLAVVFVTR-C

6. The peptide of claim 1, characterized in that the peptide has an amino acid sequence SEQ ID NO: 14
(SEQ ID NO 14)
CSFKGTTVYALSNVRSYSFVKC.

7. The peptide of claim 1, characterized in that the peptide has an amino acid sequence SEQ ID NO: 14, as indicated below:
(SEQ ID NO 15)
CSFKGTNVYALTKVRSYSFVSC.

8. The peptide of claim 1, wherein the peptide is selected from: SSKSSNLAVVFVTRCCKEYFYT (SEQ ID NO 45); SKSSNLAVVFVTRCCKEYFYTS (SEQ ID NO 46); KSSNLAVVFVTRCCKEYFYTSS (SEQ ID NO 47); SSNLAVVFVTRCCKEYFYTSSK (SEQ ID NO 48); SNLAVVFVTRCCKEYFYTSSKS (SEQ ID NO 49); NLAVVFVTRCCKEYFYTSSKSS (SEQ ID NO 50); SFKGTTVYALSNVRSYSFVKCC (SEQ ID NO 51); FKGTTVYALSNVRSYSFVKCCS (SEQ ID NO 52); SNVRSYSFVKCCSFKGTTVYAL (SEQ ID NO 53); NVRSYSFVKCCSFKGTTVYALS (SEQ ID NO 54); SYSFVKCCSFKGTTVYALSNVR (SEQ ID NO 55); YSFVKCCSFKGTTVYALSNVRS (SEQ ID NO 56); SFVKCCSFKGTTVYALSNVRSY (SEQ ID NO 57); FVKCCSFKGTTVYALSNVRSYS (SEQ ID NO 58); or a combination thereof.

9. A method of treating an inflammatory disease, disorder, condition, or symptom, comprising administering to an individual in need thereof a therapeutically-effective amount of agent that inhibits interactions between RANTES and Platelet Factor 4.

10. The method of claim 9, wherein the active agent specifically binds to the RANTES interacting domain of PF4.

11. The method of claim 9, wherein the active agent is an isolated peptide that has the amino acid sequence SEQ ID NO: 1, as indicated below:
C-X1-X2-YFYTS-X3-X4-X5-X6-X7-X8-X9-X10-X11-X12-X13-X14-X15-C
where:
X1 is chosen from the group containing lysine, glutamine, arginine, histidine and asparagine, or an amino acid deletion;
X2 is chosen from the group containing glutamic acid, aspartic acid and glutamine, or an amino acid deletion;
X3 is chosen from the group containing glycine, serine and alanine;
X4 is chosen from the group containing lysine, leucine and arginine;
X5 is chosen from the group containing serine, cysteine, glycine and threonine;
X6 is chosen from the group containing proline and alanine;
X7 is chosen from the group containing asparagine and glutamine;
X8 is chosen from the group containing proline, tyrosine and glycine;
X9 is chosen from the group containing glycine, alanine and serine;
X10 is chosen from the group containing isoleucine, valine and asparagine;
X11 is chosen from the group containing valine, isoleucine and asparagine;
X12 is chosen from the group containing phenylalanine, tyrosine, isoleucine, valine, leucine and methionine;
X13 is chosen from the group containing isoleucine, valine, leucine, methionine and phenylalanine;
X14 is chosen from the group containing threonine, glycine, alanine, serine and tyrosine;
X15 is chosen from the group containing arginine, lysine, alanine, glutamine, histidine and asparagine, or an amino acid deletion.

12. The method of claim 9, wherein the active agent is an isolated peptide that has the amino acid sequence SEQ ID NO: 2, as indicated below:
C-KEYFYTSGKCSNPAVVFVTR-C.

13. The method of claim 9, wherein the active agent is an isolated peptide that has the amino acid sequence SEQ ID NO: 3, as indicated below:
C-KEYFYTSSKCSNLAVVFVTR-C.

14. The method of claim 9, wherein the active agent is an isolated peptide that has the amino acid sequence SEQ ID NO: 4, as indicated below:
C-QEYFYTSSKCSMAAVVFITR-C.

15. The method of claim 9, wherein the active agent is an isolated peptide that has the amino acid sequence SEQ ID NO: 13, as indicated below:
(SEQ ID NO: 13)
C-KEYFYTSSKSSNLAVVFVTR-C

16. The method of claim 9, wherein the active agent is an isolated peptide that has the amino acid sequence SEQ ID NO: 14
(SEQ ID NO 14)
CSFKGTTVYALSNVRSYSFVKC.

17. The method of claim 9, wherein the active agent is an isolated peptide that has the amino acid sequence SEQ ID NO: 14, as indicated below:
(SEQ ID NO 15)
CSFKGTNVYALTKVRSYSFVSC.

18. The method of claim 9, wherein the active agent is selected from:
SSKSSNLAVVFVTRCCKEYFYT (SEQ ID NO 45); SKSSNLAVVFVTRCCKEYFYTS (SEQ ID NO 46); KSSNLAVVFVTRCCKEYFYTSS (SEQ ID NO 47); SSNLAVVFVTRCCKEYFYTSSK (SEQ ID NO 48); SNLAVVFVTRCCKEYFYTSSKS (SEQ ID NO 49); NLAVVFVTRCCKEYFYTSSKSS (SEQ ID NO 50); SFKGTTVYALSNVRSYSFVKCC (SEQ ID NO 51); FKGTTVYALSNVRSYSFVKCCS (SEQ ID NO 52); SNVRSYSFVKCCSFKGTTVYAL (SEQ ID NO 53); NVRSYSFVKCCSFKGTTVYALS (SEQ ID NO 54); SYSFVKCCSFKGTTVYALSNVR (SEQ ID NO 55); YSFVKCCSFKGTTVYALSNVRS (SEQ ID NO 56); SFVKCCSFKGTTVYALSNVRSY (SEQ ID NO 57); FVKCCSFKGTTVYALSNVRSYS (SEQ ID NO 58); or a combination thereof.

19. The method of claim 9, further comprising a second active agent that treats an inflammatory disease, disorder, condition, or symptom.

20. The method of claim 9, wherein the inflammatory disease, disorder or condition is Atherosclerosis; Abdominal aortic aneurysm (AAA) disease; Acute disseminated encephalomyelitis; Moyamoya disease; Takayasu disease; Acute coronary syndrome; Cardiac-allograft vasculopathy; Pulmonary inflammation; Acute respiratory distress syndrome; Pulmonary fibrosis; Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome; Multiple sclerosis; Myasthenia gravis; Myocarditis; Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder; Endotoxin shock; Septic shock; Rheumatoid spondylitis; Ankylosing spondylitis; Gouty arthritis; Polymyalgia rheumatica; Alzheimer's disorder; Parkinson's disorder; Epilepsy; AIDS dementia; Asthma; Adult respiratory distress syndrome; Bronchitis; Cystic fibrosis; Acute leukocyte-mediated lung injury; Distal proctitis; Wegener's granulomatosis; Fibromyalgia; Bronchitis; Uveitis; Conjunctivitis; Psoriasis; Eczema; Dermatitis; Smooth muscle proliferation disorders; Meningitis; Shingles; Encephalitis; Nephritis; Tuberculosis; Retinitis; Atopic dermatitis; Pancreatitis; Periodontal gingivitis; Coagulative Necrosis; Liquefactive Necrosis; Fibrinoid Necrosis; Neointimal hyperplasia; Myocardial infarction; Stroke; organ transplant rejection; influenza, or combinations thereof.

21. A method of treating a disorder of a cardiovascular system, comprising co-administering to an individual in need thereof a synergistic combination of (a) a therapeutically-effective amount of an agent that inhibits the interaction between RANTES and Platelet Factor 4; and (b) a second active agent selected from an agent that treats a cardiovascular disorder.

22. The method of claim 21, wherein administration of the second active agent partially or fully results in undesired inflammation.

23. The method of claim 21, wherein the second active agent is niacin; a fibrate; a statin; an apolipoprotein A-1 modulator; an ACAT modulator; a CETP modulator; a glycoprotein IIb/IIIa modulator; a P2Y12 modulator; an Lp-PLA2 modulator; an anti-hypertensive; a leukotriene inhibitor; an 5-LO inhibitor; a FLAP inhibitor; or combinations thereof.

24. The method of claim 21, wherein the disorder is hyperlipidemia; hypercholesterolemia; hyperglyceridemia; combined hyperlipidemia; hypolipoproteinemia; hypocholesterolemia; abetlipoproteinemia; Tangier disease; acute coronary syndrome; unstable angina; non-ST segment elevation myocardial infarction; ST segment elevation myocardial infarction; stable angina; Prinzmetal's angina; arteriosclerosis; atherosclerosis; arteriolosclerosis; stenosis; restenosis; venous thrombosis; arterial thrombosis; stroke; transient ischemic attack; peripheral vascular disease; coronary artery disease; hypertension; or combinations thereof.


SEQUENCE LISTING (96 sequences)

SEQ ID NO: 1 - protein, 22 aa, Artificial Sequence
  Description of Artificial Sequence Synthetic peptide
CXXYFYTSXXXXXXXXXXXXXC

SEQ ID NO: 2 - protein, 22 aa, Artificial Sequence
  Description of Artificial Sequence Synthetic peptide
CKEYFYTSGKCSNPAVVFVTRC

SEQ ID NO: 3 - protein, 22 aa, Artificial Sequence
  Description of Artificial Sequence Synthetic peptide
CKEYFYTSSKCSNLAVVFVTRC

SEQ ID NO: 4 - protein, 22 aa, Artificial Sequence
  Description of Artificial Sequence Synthetic peptide
CQEYFYTSSKCSMAAVVFITRC

SEQ ID NO: 5 - protein, 22 aa, Artificial Sequence
  Description of Artificial Sequence Synthetic peptide
CXXYFYTSXXXXXXXXXXXXXC

SEQ ID NO: 6 - protein, 22 aa, Artificial Sequence
  Description of Artificial Sequence Synthetic peptide
CXXYFYTSXXXXXXXXXXXXXC

SEQ ID NO: 7 - protein, 22 aa, Artificial Sequence
  Description of Artificial Sequence Synthetic peptide
CXXYFYTSXXXXXXXXXXXXXC

SEQ ID NO: 8 - protein, 22 aa, Artificial Sequence
  Description of Artificial Sequence Synthetic peptide
CXXYFYTSXXXXXXXXXXXXXC

SEQ ID NO: 9 - protein, 22 aa, Artificial Sequence
  Description of Artificial Sequence Synthetic peptide
CXXYFYTSXXXXXXXXXXXXXC

SEQ ID NO: 10 - protein, 22 aa, Artificial Sequence
  Description of Artificial Sequence Synthetic peptide
CXXYFYTSXXXXXXXXXXXXXC

SEQ ID NO: 11 - protein, 22 aa, Artificial Sequence
  Description of Artificial Sequence Synthetic peptide
CXXYFYTSXXXXXXXXXXXXXC

SEQ ID NO: 12 - protein, 22 aa, Artificial Sequence
  Description of Artificial Sequence Synthetic peptide
CXXYFYTSXXXXXXXXXXXXXC

SEQ ID NO: 13 - protein, 22 aa, Artificial Sequence
  Description of Artificial Sequence Synthetic peptide
CKEYFYTSSKSSNLAVVFVTRC

SEQ ID NO: 14 - protein, 22 aa, Artificial Sequence
  Description of Artificial Sequence Synthetic peptide
CSFKGTTVYALSNVRSYSFVKC

SEQ ID NO: 15 - protein, 22 aa, Artificial Sequence
  Description of Artificial Sequence Synthetic peptide
CSFKGTNVYALTKVRSYSFVSC

SEQ ID NO: 16 - protein, 20 aa, Homo sapiens
KEYFYTSGKCSNPAVVFVTR

SEQ ID NO: 17 - protein, 16 aa, Pan troglodytes
KEYFYTSGKCSNPAVV

SEQ ID NO: 18 - protein, 20 aa, Pongo pygmaeus
KEYFYTSGKCSNPAVVFVTR

SEQ ID NO: 19 - protein, 20 aa, Macaca mulatta
KEYFYTSGKCSNPAVVFVTR

SEQ ID NO: 20 - protein, 20 aa, Otolemur garnettii
KEYFYTSGKCSNPAVVFITR

SEQ ID NO: 21 - protein, 20 aa, Microcebus murinus
MEYFYTSGKCSNPAVVFITR

SEQ ID NO: 22 - protein, 20 aa, Ochotona princeps
KEYFYTSGKCSNPAVVFVTR

SEQ ID NO: 23 - protein, 20 aa, Oryctolagus cuniculus
TEYFYTSGKCSFPAVVFVTR

SEQ ID NO: 24 - protein, 20 aa, Mus musculus
KEYFYTSSKCSNLAVVFVTR

SEQ ID NO: 25 - protein, 20 aa, Rattus norvegicus
KEYFYTSSKCSNLAVVFVTR

SEQ ID NO: 26 - protein, 20 aa, Peromyscus maniculatus
KEYFYTSSKCSNSAVVFVTR

SEQ ID NO: 27 - protein, 20 aa, Sigmodon hispidus
KEYFYTSSKCSNFAVVFVTR

SEQ ID NO: 28 - protein, 20 aa, Cavia porcellus
KEYFYTSSKCSNLAVVFVTR

SEQ ID NO: 29 - protein, 15 aa, Spermophilus tridecemlineatus
KEYFYTSSKCSNLAV

SEQ ID NO: 30 - protein, 20 aa, Felis catus
QEYFYTSSKCSMPAVVFVTR

SEQ ID NO: 31 - protein, 20 aa, Canis familiaris
QEYFYTSSKCSMPAVVFVTR

SEQ ID NO: 32 - protein, 20 aa, Sus scrofa
QEYFYTSSKCSMAAVVFITR

SEQ ID NO: 33 - protein, 20 aa, Bos taurus
QEYFYTSSKCSMAAVVFITR

SEQ ID NO: 34 - protein, 20 aa, Equus caballus
QEYFYTSSKCSIPAVVFVTR

SEQ ID NO: 35 - protein, 20 aa, Monodelphis domestica
REYFYTSSRCGNLGVVFITR

SEQ ID NO: 36 - protein, 15 aa, Loxodonta africana
KEYFYTSGKCSMPAV

SEQ ID NO: 37 - protein, 15 aa, Dasypus novemcinctus
KEYFYTSGKCSNPAV

SEQ ID NO: 38 - protein, 20 aa, Echinops telfairi
REYFYTSSKCTSPAVVFVTR

SEQ ID NO: 39 - protein, 21 aa, Erinaceus europaeus
QEYFYTSSKCSIPSAVVFVTR

SEQ ID NO: 40 - protein, 20 aa, Tupaia belangeri
REYFYTSGKCSNPAVVFITR

SEQ ID NO: 41 - protein, 20 aa, Sorex araneus
QDYFYTSSKCSMPAVVFVTR

SEQ ID NO: 42 - protein, 20 aa, Gallus gallus
KDYFYTSSKCPQAAVVFITR

SEQ ID NO: 43 - protein, 20 aa, Anas platyrhynchos
KDYFYTSSKCPQPAVVFITR

SEQ ID NO: 44 - protein, 20 aa, Myotis lucifugus
QEYFYTSSKCSMPAVVLITR

SEQ ID NO: 45 - protein, 22 aa, Artificial Sequence
  Description of Artificial Sequence Synthetic peptide
SSKSSNLAVVFVTRCCKEYFYT

SEQ ID NO: 46 - protein, 22 aa, Artificial Sequence
  Description of Artificial Sequence Synthetic peptide
SKSSNLAVVFVTRCCKEYFYTS

SEQ ID NO: 47 - protein, 22 aa, Artificial Sequence
  Description of Artificial Sequence Synthetic peptide
KSSNLAVVFVTRCCKEYFYTSS

SEQ ID NO: 48 - protein, 22 aa, Artificial Sequence
  Description of Artificial Sequence Synthetic peptide
SSNLAVVFVTRCCKEYFYTSSK

SEQ ID NO: 49 - protein, 22 aa, Artificial Sequence
  Description of Artificial Sequence Synthetic peptide
SNLAVVFVTRCCKEYFYTSSKS

SEQ ID NO: 50 - protein, 22 aa, Artificial Sequence
  Description of Artificial Sequence Synthetic peptide
NLAVVFVTRCCKEYFYTSSKSS

SEQ ID NO: 51 - protein, 22 aa, Artificial Sequence
  Description of Artificial Sequence Synthetic peptide
SFKGTTVYALSNVRSYSFVKCC

SEQ ID NO: 52 - protein, 22 aa, Artificial Sequence
  Description of Artificial Sequence Synthetic peptide
FKGTTVYALSNVRSYSFVKCCS

SEQ ID NO: 53 - protein, 22 aa, Artificial Sequence
  Description of Artificial Sequence Synthetic peptide
SNVRSYSFVKCCSFKGTTVYAL

SEQ ID NO: 54 - protein, 22 aa, Artificial Sequence
  Description of Artificial Sequence Synthetic peptide
NVRSYSFVKCCSFKGTTVYALS

SEQ ID NO: 55 - protein, 22 aa, Artificial Sequence
  Description of Artificial Sequence Synthetic peptide
SYSFVKCCSFKGTTVYALSNVR

SEQ ID NO: 56 - protein, 22 aa, Artificial Sequence
  Description of Artificial Sequence Synthetic peptide
YSFVKCCSFKGTTVYALSNVRS

SEQ ID NO: 57 - protein, 22 aa, Artificial Sequence
  Description of Artificial Sequence Synthetic peptide
SFVKCCSFKGTTVYALSNVRSY

SEQ ID NO: 58 - protein, 22 aa, Artificial Sequence
  Description of Artificial Sequence Synthetic peptide
FVKCCSFKGTTVYALSNVRSYS

SEQ ID NO: 59 - protein, 18 aa, Artificial Sequence
  Description of Artificial Sequence Synthetic peptide
DWFKAFYDKVAEKFKEAF

SEQ ID NO: 60 - protein, 20 aa, Artificial Sequence
  Description of Artificial Sequence Synthetic peptide
CXXXXXTXXXXSNXXXXXXX

SEQ ID NO: 61 - protein, 19 aa, Artificial Sequence
  Description of Artificial Sequence Synthetic peptide
CXXXXXTXXXXSNXXXXXX

SEQ ID NO: 62 - protein, 18 aa, Artificial Sequence
  Description of Artificial Sequence Synthetic peptide
CXXXXXTXXXXSNXXXXX

SEQ ID NO: 63 - protein, 17 aa, Artificial Sequence
  Description of Artificial Sequence Synthetic peptide
CXXXXXTXXXXSNXXXX

SEQ ID NO: 64 - protein, 16 aa, Artificial Sequence
  Description of Artificial Sequence Synthetic peptide
CXXXXXTXXXXSNXXX

SEQ ID NO: 65 - protein, 15 aa, Artificial Sequence
  Description of Artificial Sequence Synthetic peptide
CXXXXXTXXXXSNXX

SEQ ID NO: 66 - protein, 14 aa, Artificial Sequence
  Description of Artificial Sequence Synthetic peptide
CXXXXXTXXXXSNX

SEQ ID NO: 67 - protein, 13 aa, Artificial Sequence
  Description of Artificial Sequence Synthetic peptide
CXXXXXTXXXXSN

SEQ ID NO: 68 - protein, 21 aa, Artificial Sequence
  Description of Artificial Sequence Synthetic peptide
XXXXXTXXXXSNXXXXXXXKC

SEQ ID NO: 69 - protein, 20 aa, Artificial Sequence
  Description of Artificial Sequence Synthetic peptide
XXXXTXXXXSNXXXXXXXKC

SEQ ID NO: 70 - protein, 19 aa, Artificial Sequence
  Description of Artificial Sequence Synthetic peptide
XXXTXXXXSNXXXXXXXKC

SEQ ID NO: 71 - protein, 18 aa, Artificial Sequence
  Description of Artificial Sequence Synthetic peptide
XXTXXXXSNXXXXXXXKC

SEQ ID NO: 72 - protein, 17 aa, Artificial Sequence
  Description of Artificial Sequence Synthetic peptide
XTXXXXSNXXXXXXXKC

SEQ ID NO: 73 - protein, 16 aa, Artificial Sequence
  Description of Artificial Sequence Synthetic peptide
TXXXXSNXXXXXXXKC

SEQ ID NO: 74 - protein, 15 aa, Artificial Sequence
  Description of Artificial Sequence Synthetic peptide
XXXXSNXXXXXXXKC

SEQ ID NO: 75 - protein, 14 aa, Artificial Sequence
  Description of Artificial Sequence Synthetic peptide
XXXSNXXXXXXXKC

SEQ ID NO: 76 - protein, 13 aa, Artificial Sequence
  Description of Artificial Sequence Synthetic peptide
XXSNXXXXXXXKC

SEQ ID NO: 77 - protein, 12 aa, Artificial Sequence
  Description of Artificial Sequence Synthetic peptide
XSNXXXXXXXKC

SEQ ID NO: 78 - protein, 11 aa, Artificial Sequence
  Description of Artificial Sequence Synthetic peptide
SNXXXXXXXKC

SEQ ID NO: 79 - protein, 13 aa, Artificial Sequence
  Description of Artificial Sequence Synthetic peptide
CXXXXXTXXXXSN

SEQ ID NO: 80 - protein, 16 aa, Artificial Sequence
  Description of Artificial Sequence Synthetic peptide
TXXXXSNXXXXXXXKC

SEQ ID NO: 81 - protein, 15 aa, Artificial Sequence
  Description of Artificial Sequence Synthetic peptide
XXXXSNXXXXXXXKC

SEQ ID NO: 82 - protein, 14 aa, Artificial Sequence
  Description of Artificial Sequence Synthetic peptide
XXXSNXXXXXXXKC

SEQ ID NO: 83 - protein, 13 aa, Artificial Sequence
  Description of Artificial Sequence Synthetic peptide
XXSNXXXXXXXKC

SEQ ID NO: 84 - protein, 12 aa, Artificial Sequence
  Description of Artificial Sequence Synthetic peptide
XSNXXXXXXXKC

SEQ ID NO: 85 - protein, 11 aa, Artificial Sequence
  Description of Artificial Sequence Synthetic peptide
SNXXXXXXXKC

SEQ ID NO: 86 - protein, 22 aa, Artificial Sequence
  Description of Artificial Sequence Synthetic peptide
CKEYFYTSSKSSNLAVVFVTRC

SEQ ID NO: 87 - protein, 9 aa, Artificial Sequence
  Description of Artificial Sequence Synthetic peptide
KEYFYTSGK

SEQ ID NO: 88 - protein, 22 aa, Artificial Sequence
  Description of Artificial Sequence Synthetic peptide
CKEYFYTSXXXXXXXXXXXXXC

SEQ ID NO: 89 - protein, 22 aa, Artificial Sequence
  Description of Artificial Sequence Synthetic peptide
CKEYFYTSXXXXXXXXVFXTRC

SEQ ID NO: 90 - protein, 22 aa, Artificial Sequence
  Description of Artificial Sequence Synthetic peptide
CKEYFYTSXXXXXXXXVFVTRC

SEQ ID NO: 91 - protein, 22 aa, Artificial Sequence
  Description of Artificial Sequence Synthetic peptide
CKEYFYTSSXXXXLAVVFVTRC

SEQ ID NO: 92 - protein, 22 aa, Artificial Sequence
  Description of Artificial Sequence Synthetic peptide
CKEYFYTSXXXXXXXXXXXXXC

SEQ ID NO: 93 - protein, 22 aa, Artificial Sequence
  Description of Artificial Sequence Synthetic peptide
CKEYFYTSXXXXXXXXVFXTRC

SEQ ID NO: 94 - protein, 22 aa, Artificial Sequence
  Description of Artificial Sequence Synthetic peptide
CKEYFYTSXXXXXXXXVFITRC

SEQ ID NO: 95 - protein, 22 aa, Artificial Sequence
  Description of Artificial Sequence Synthetic peptide
CKEYFYTSGXXXXPGIVFITRC

SEQ ID NO: 96 - protein, 22 aa, Artificial Sequence
  Description of Artificial Sequence Synthetic peptide
CKEYFYTSGKSSNPGIVFITRC